(19) United States
(12) Patent Application Publication
(10) Pub. No.: US 2024/0384291 A1
(43) Pub. Date: Nov. 21, 2024

(54) Effective Interventions With Aging and Diseases Of Aging Of Human and Their Consequences

(71) Applicant: Sinan Tas, Bor (TR)

(72) Inventor: Sinan Tas, Bor (TR)

(21) Appl. No.: 18/445,718

(22) PCT Filed: Sep. 13, 2021
(86) PCT No.: PCT/TR2021/000001

§ 371 Date: Dec. 28, 2023

(51) Int. Cl. C12N 15/85; A61K 35/54

(52) U.S. Cl. CPC C12N 15/85; A61K 35/54

(57) ABSTRACT

The diseases and disabilities associated with aging are at roots of growing unsolved problems in human societies worldwide and the basic problem is inherent in human genome and biology. Increases of average human lifespan without a decrease of rate of aging have been causing increases in frequencies of the diseases of aging whose symptomatic treatments have limited benefits. Here it is shown with exemplification that identifications and targeting of the decisive upstream mechanisms of biological aging and of age associated diseases can provide effective solutions to the problem.

### TECHNICAL FIELD

Invention and previously undescribed findings in biological-medical sciences and technologies are presented concerning solutions of previously unsolved problems associated with aging of human.

### BACKGROUND OF THE INVENTION

Average human lifespan has reached to historic highs in industrialized societies in parallel with effective treatments and preventions of former common killers like bacterial infections. These increases of average human lifespan did not happen through a decrease of rate of biological aging as testified by the absence of a detectable change in the maximum lifespan potential (MLP) of human, which primarily is a genetically affected trait. In accord a distinct group of age-associated diseases have now become frequent worldwide, particularly in industrialized countries. Problems caused by these typically chronic diseases to patients, to their families and to society are compounded by the fact that currently practiced treatments are mostly symptomatic, providing not more than alleviation of persistent or recurring symptoms. Not even palliation is available for some frequent age-associated diseases and the affected patients commonly experience decreases/losses of working ability and high costs of their conditions' managements and the age-associated diseases have become a mounting burden in human societies through direct and indirect effects (1-3) (publications that are referenced herein are identified by numbers in brackets and listed at the end of description in numerical order of citation).

Poor countries where preventable causes of death are not effectively dealt with and have high birth rates may be little touched as yet by the diseases of aging but the present situation in them does not represent a solution. Not only their economic developments would lead to disappearances of preventable causes of death and increases of average lifespan but high birth rates and increases of population size would eventually collide with the realities that world cannot sustain a human population beyond a limit, that some effects of even current populations are not containable within national borders and that the global environmental effects are likely to increase with their industrializations. Expanding the population size cannot therefore solve the problems that have been arising from the increases of human lifespan without a slowing of biological aging. Thus the humankind faces an unsolved problem that for the first time in history has started to have significant impact on societies worldwide.

Question of whether the rate of biological aging can be slowed in human must therefore be answered. Fossil records, genome analyses and other findings showing that during the approximately six million years between the occurrence of earliest hominids and modern humans the MLP increased significantly (4, 5) may be considered an affirmative answer. Such evolution cannot however be relied upon for a solution of the problem faced at present. Not only the upper limit of human population sustainable by world would be reached within a much smaller timescale, but the easier genetic changes that have provided the extension of MLP have already taken place in the human genome. Further in this respect, molecular genetic methods that allow desired changes in genomes of laboratory animals show that random changing of more than a few genes simultaneously often prove lethal in reminder of the fact that present day genomes evolved over hundreds of millions of years. More fundamentally, there are basic biological reasons why classical evolutionary forces cannot provide an effective solution to the aforementioned problem arising from aging and age-associated diseases (6). Investigations into mechanisms of aging have on the other hand revealed also examples of causation of slowing of aging in laboratory animals. Thus, whereas the usual therapeutic approaches of attempting to treat each disease of aging in isolation have not in most cases provided benefits beyond the symptomatic, the few environmental and genetic modifications that enable even modest (˜20%) increases of MLP in laboratory animals have shown provision of across the board decreases and delaying of most age-associated diseases and shown occurrences of healthier animals at advanced ages when their non-intervened counterparts had either died or had become moribund with diseases of aging (e.g. references 7-9 and references in them).

### SUMMARY OF THE INVENTION

The present invention concerns slowing of the rate of aging of human and prevention and treatments of the diseases associated with aging of human.

In one aspect, the invention concerns changing of nucleotide sequences in human genome that provides increases of lifespans of functionally competent normal somatic tissue cells and decreases of occurrences of senescence of cells in tissues and organs.

In a further aspect, generation of universally histocompatible genetically engineered normal cells of human for transplantation to a desired tissue site in a human subject for slowing of rate of aging of the subject and for prevention and treatments of disorders of aging is described and avoidance of destruction of industrially produced such cells by natural killer cells of transplanted persons is described.

Other features and embodiments of the invention and of the related novel findings that are presented herein are evident to scientists skilled in the areas of the invention from the detailed description that follows below.

### DETAILED DESCRIPTION OF THE INVENTION

The previously undescribed findings that relate to the invention described herein include those that are interdisciplinary. In addition, effective interventions with aging and age-associated diseases of human have social, international, economic impacts and their implementations are affected by such factors. Therefore to facilitate the presentation to readers having diverse backgrounds, the descriptions below include mentions of common knowledge of specialists in biological-medical disciplines that may not be common knowledge for a specialist in another discipline. Referencing to scientific publications pertaining to a particular subject is in general to publications that are representative rather than inclusive and technical manuals and textbook knowledge are in general not cited since such knowledge is considered to be part of the common general knowledge of those skilled in the technical fields of the presented invention.

Aging and age-associated diseases of human represent complex processes from molecular-cellular levels to levels of tissues and organs and at whole organism level. I have analyzed these and point herein to the decisive upstream mechanisms of aging and of age-associated diseases of human and to interventions with them.

### Relevant Basics of Human Biology and Conventional Medical Practice

Features of human organism are determined to a large extend by human genome. Environmental variables experienced during embryonal-fetal development, childhood and afterwards affect expressions of that genetic information and contribute to the phenotype of each person. Human genome is broadly similar or identical between any two men or women living in different countries around world for most of the genome (at ˜99.9% of nucleotide sequences) in accord with the common ancestors of present day people (10). On the other hand ˜0.1% of the ˜3 billion nucleotides of genetic information inherited from mother and ˜3 billion from father corresponds to a large number and every person is genetically unique. Because chromosomes can undergo nucleotide sequence changes by recombination and by further mechanisms during formation of germ cells, and because which particular copy of the two homologous chromosomes is acquired by a given oocyte or sperm (and then which two are fused) are essentially random occurrences, children of same parents have differing genotypes. In addition somatic cells are exposed to varying environmental and endogenous damaging agents that can cause varying changes of nucleotide sequences and of gene expression. Thus basic human biology makes each person unique genetically and phenotypically.

Studies of aging and of age-associated diseases of human have shown complex processes. Countless age-associated changes in structure and function of virtually every cell type, tissue, organ and system have been described on top of those at molecular level. Decreases of physiological capabilities are found with increasing chronological age of adults at varying rates in different individuals but age-associated declines are seen in some physiological functions already starting during childhood. Distinct age-associated diseases affecting one or more organs become detectable during aging of human. Treatments of many are at present symptomatic and while alleviations or temporary disappearances of symptoms can help, reappearances of symptoms and typical additions of further age-associated diseases in aging patients have led to conclusions of helplessness and settlements to palliation (e.g. reference 11).

### Upstream Mechanisms Driving Complex Processes of Aging

Whereas aging is a complex process affected by supracellular interactions, several lines of evidence indicate that aging of organism arises primarily from intrinsic age-associated failures of cells (12). Experimental findings and comparative analyses have revealed particular upstream events that are instrumental in cellular aging and aging of organism across species. The answer to the question whether or not there are life forms that do not show aging is affirmative and I have pointed to the unifying features of those that do not show aging and those that undergo aging (12). First, prokaryotes do not show the aging and limited clonal lifespan exhibited by cells of eukaryotic organisms. Even the unicellular eukaryotes show limitation of clonal lifespan and exhibit characteristic morphological and molecular changes near end of lifespan that are similar to those seen in somatic cells of human at old age. In avoiding extinction that would otherwise occur, unicellular eukaryotes undergo periodic rejuvenation through meiosis similar to the meiotic rejuvenation in multicellular eukaryotes and the basic mechanisms of meiotic rejuvenation are largely conserved among eukaryotes (12). Meiotic rejuvenation in human can be recognized by considering that oocytes and sperm can give rise to youthful children even when from a woman nearing menopause and from a man beyond average human lifespan.

Prokaryotes and eukaryotes show extensive similarities of their molecular constituents, biochemical reactions and genome sequences in accord with the evolution of latter from former. Geological and fossil records and genome analyses reveal occurrence of eukaryotes from ancient symbiosis events about two billion years ago and to origination of mitochondria around the time when the atmosphere of earth became oxidizing as it is today (13). Mitochondrial oxidative energy metabolism provides much more ATP for energy demanding life processes than available otherwise and had conferred advantages in a world with an oxidizing atmosphere. On the other hand radicals and prooxidant molecules generated by oxidative metabolism cause damaging of nucleic acids, proteins and other cellular constituents and the oxidatively damaged macromolecules are found at increasing amounts in various somatic cells with increasing age of organism. Effective prevention and repairing of these damages positively correlate with the species-specific MLP. Oxidative metabolism and prooxidants do not however suffice to account for aging. For example, there are prokaryotes thriving in strongly oxidizing media without limitation of clonal lifespan and they show efficient repair of DNA double strand breaks and other damages that occur in such media in amounts magnitudes above the minimal doses lethal for eukaryotes (14). I have pointed in this respect to a fundamental problem that is inherent in structure of the eukaryotic genetic material and is upstream in aging process (12). Whereas the primary structure of DNA is likewise in prokaryotes and eukaryotes, eukaryotic DNA is complexed with histones and further particular proteins to create the chromatin complex in which accessibility of DNA is highly restricted unlike the situation in prokaryotes. The prokaryotic genome is in addition typically circular DNA unlike the linear chromosomal DNA of eukaryotes which causes challenges of replication of ends of linear DNA molecules but it is not a primary or determinative factor in aging as pointed below. The regulated limitation of access to DNA enabled by the chromatin structure allows inheritable generation of phenotypically different cell types having the same genome and it is a key requirement for cellular differentiation. Cellular differentiation paved the way to evolution of advanced multicellular eukaryotic organisms but the restriction of accessibility of particular regions of DNA by packaging into a compact structure of chromatin (heterochromatin) that occurs during cellular differentiation has costs in terms of repair of damage to the genetic material and in terms of aging (12).

Cancer is a disease associated with aging. The tumorigenic cells in tumors show in general less differentiation or blocking of differentiation in comparison to their normal counterparts in the tissues they are found. They also have unlimited lifespan potential in vivo (demonstrable by serial transplantation in histocompatible inbred animals) and in vitro while their normal counterparts show limited clonal lifespan under the same conditions and show typical molecular and morphological signs of senescence near end of lifespan. Towards determination of mechanisms of tumorigenesis during aging and of acquirement of infinite lifespan potential by tumor cells, analyses of the chromatin complex have been carried out at levels from nucleosomal to the intact complex existing in cell nucleus in normal tissue cells and in their neoplastic counterparts in aging human and other species. The findings revealed that cancer cells consistently avoid a particular subset of the structural alterations of chromatin that occur in normal somatic cells during aging both in human and mice (6, 12, 15). Specifically, treatment of the demembranized cell nucleus or of the complexes of nuclear DNA loops anchored at nuclear matrix-lamina with disulfide reducing agents caused their decondensation to a greater extend when prepared from normal somatic cells of older mice and human in comparison to those of young adult ages (6, 12, 16-18) while the neoplastic counterparts of studied normal cells have shown consistently less to undetectable decondensation (6, 12, 19). Controls and measures against artefactual oxidation-reduction reactions of sulfhydryl-disulfide groups during processing of tissues starting with live tissue cells have shown that the observed effects of aging and of neoplastic transformation reflect the in vivo situation.

Neoplastic cells have not on the other hand shown avoidance or reversion of another age-associated modification of chromatin revealed by an age-associated increase in the accessibility of DNA to the added endonucleases in a constitutive heterochromatin enriched fraction containing ˜70% or more of the nuclear DNA in the same cells that showed an age-associated increase in the disulfide mediated condensation of chromatin (6, 16, 17), emphasizing that neoplastic transformation does not provide a genuine reversion to a youthful cellular phenotype unlike the situation with meiotic rejuvenation. In accord, maintenance of genetic stability in neoplastic cells is even worse than in the normal cells of old animals and this has relevance for safe and effective treatment of tumor bearing human (pointed below).

Heterochromatinization of select regions of DNA during differentiation of cells from stem cells towards terminally differentiated progeny (facultative heterochromatinization) provides suppression of expression of the genetic information in the selected regions and shares features and mechanisms with the constitutive heterochromatinization that helps to suppress expressions of transposable elements (TE's). Constitutive heterochromatin occurs at identical or nearly identical regions of genome in different cell types of multicellular eukaryotes, mostly around TE's and other repeated sequences, and the compaction of chromatin around these sequences is among the earliest events of embryogenesis (20-22). Relatively more of diverse TE's appear to have undergone inactivating mutations during evolution of human in comparison to a shorter-living mammalian (23). Truncated and otherwise inactivated TE's and the sequences derived from them continue on the other hand to exist in human genome and make a much greater part of it than the sequences that encode proteins. Some of the TE-derived sequences have been co-opted for regulations of host genes and currently active TE's include those having effects on early development (20, 24) but a large proportion of the constitutively heterochromatinized sequences in human genome is dispensable, lacking essential function and having rather negative effects on healthy lifespan as pointed below.

Damages to the genetic material in heterochromatinized regions of genome pose problems directly relevant to aging. Not only the heterochromatin in a damaged region must be opened up to provide access of repair enzymes and of other repair proteins to a site of damage (which creates risks of access also by unwanted enzymes and unwanted modifications) but the structure of chromatin therein must be reinstituted to the pre-damaged state even when a DNA repair is successful. Earlier findings had revealed failures in this respect during aging (12).

### Critical Mechanisms of Meiotic Rejuvenation

Details of the mechanisms by which germ line cells are capable of giving rise to youthful organisms at ages beyond the average lifespan of organism would facilitate effective interventions with disorders of aging. These have been coming to light in diverse eukaryotic species investigated for various purposes. Primordial germ cells (PGC's) that give rise to the oocyte and sperm are specified early during embryonal development and are found to show extensive decondensation of chromatin and near complete demethylation of DNA in association with active repair of DNA, except for the DNA of a subset of the currently activatable TE's (25-27). These cells have also upregulated expressions of DNA repair enzymes and of other factors that participate in various forms of DNA repair and the decondensation of chromatin in them serves for efficient repair of DNA. Oocytes and spermatocytes have further advantages as well during meiosis for repairs of DNA damages. The attachments of homologous chromosomes to nuclear lamina-envelope side-by-side during prophase I of meiosis (“bouquet” at zygotene) facilitate the repairs via homologous recombination (HR). Apart from the usually emphasized contribution of cross-overs to generation of genetic diversity, it can be the only means of supply of genetic information when both strands of DNA in one of the homologues have undergone damages that preclude retrieval of the genetic information from complementary strand. Oocytes have also efficient means of prevention of damages to the genetic material besides for the repairing of damages in both maternal and paternal genomes when an oocyte is fertilized by a sperm. On top of the efficient means of repairing of damages to genetic material, germ cells are found to have quality control mechanisms for eliminations of those that have still retained and/or have acquired critical damages (28). As a result of such quality control, the oocytes that have progressed through prophase I of meiosis to become arrested at its end are found to be normally eliminated in high proportions by apoptosis (28).

While having similar strategies and mechanisms directed at repair and maintenance of genome, male and female germline cells show also differences which however are compatible with or conducive to the provision of a youthful organism following fertilization of oocyte by sperm. The X and Y chromosomes have no homologues in cells of males. In accord spermatocytes show the persistent DNA damage marker γH2AX in their X and Y chromatin when such had disappeared from autosomes at pachytene-leptotene of meiosis where a condensed chromatin mass (XY body) is formed in contrast to the highly decondensed chromatin of autosomes in the same cells (29). Male germline cells do not enter meiosis until puberty. They are arrested at prespermatogonia stage during development in utero, start to enter to meiosis at puberty and are capable of producing sperm lifelong in adults. Following the repair events during meiosis prophase I, the chromatin in round spermatids forming upon completions of the meiotic cell divisions show significant condensation and the sperm derived from them show further condensation and remodeling of chromatin with replacements of nucleosomal histones in most (but not all) regions of genome with protamines that undergo extensive intermolecular disulfide bonding. The female germline cells in human and other mammalians on the other hand enter to meiosis and complete its prophase I already during development in utero and are then arrested until nearing puberty. The first meiotic division occurs just before ovulation in the sexually mature female to give rise to one diploid oocyte and to one discarded diploid nucleus (first polar body) and the second meiotic division occurs in matured oocyte to give rise to one retained haploid nucleus and to one discarded (second polar body). No new immature oocyte is added postnatally to those generated during development in utero. Oocytes are in accord endowed with powerful means of preservation of genome integrity during their long rest in ovaries, which can be up to several decades in human, and oocyte provides such support also to the male genome following fertilization by sperm.

Entry of sperm nucleus into cytoplasm of mature oocyte triggers a series of reactions which cause decondensation and remodeling of both oocyte and sperm chromatin. The chromatin remodelers, reductive and proteolytic enzymes, reduced glutathione (GSH), other reducing factors and demethylaters of DNA supplied by the oocyte provide removal of protamines from paternal genome, erasure of most of its 5-methylcytosine (5mC) modifications, de novo formations of nucleosomes and active repairs of both paternal and maternal genomes shortly following fertilization (30, 31). Activations of base excision repair (BER) enzymes and their localizations to paternal and maternal pronuclei are observed and excision of a major product of oxidative DNA damage, 8-hydroxy-2′-deoxyguanosine (OxoG), is stimulated upon fertilization (31). Thus, besides minimizing or avoiding oxidative energy metabolism and providing a relatively reduced redox environment to the genetic material during the long arrest of immature oocytes (decades in human), oocytes appear to undergo BER and further repairs also following fertilization and to provide such also to the paternal genome. Despite having repairs in PGC's and at meiosis prophase I, paternal genome is susceptible to acquirements of oxidative damage due to the highly condensed chromatin of sperm and reliance of sperm on respiring mitochondria for the reach of sperm nucleus into oocyte cytoplasm. Sperm mitochondria do not normally enter into oocyte and the oocyte, source of the mitochondria of embryo, shows increased uses of the reductive power of GSH following fertilization (31). It can act against oxidative damage and mutations in nuclear and mitochondrial DNA in the early pluripotent cells of embryo from which the tissue stem cells and thereby differentiated cells of tissues and organs are derived.

The decondensation of chromatin in germline cells creates on the other hand also risks of activations of currently activatable TE's. They are accordingly targeted by multiple defenses in germline and in the early embryo cells that also show extensive decondensation of chromatin (20, 22, 30). In addition to the selective de novo DNA methylations of particular TE's that can tag them for selective heterochromatinization and selective compaction of chromatin of other TE's in the absence of methylation of their DNA, targeting of TE transcripts by RNA-based mechanisms is also used in spermatocytes and oocytes (28, 32). Physical compaction of chromatin of selected sequences can be achieved in the absence of DNA methylation by posttranslational modifications of nucleosomal histones, particularly by H3K9me3 and further modifications of H3 recognized and bound by HP1α and other heterochromatin proteins, by linker histone H1 and by further means and are observed both in germline and soma (21, 29). Repressions of TE's by H3K9me3 modification of their chromatin during early development to morula-blastocyst (21) where the genome shows the lowest 5mC content for soma and the significant compaction of chromatin during development to the 8-cell stage embryos (22), while extensive genome-wide DNA demethylation is underway, are examples of 5mC-independent means of chromatin compaction. During later development recruitments of DNA methyl transferases by HP1α and other heterochromatin proteins to H3K9me3 modified regions of chromatin can facilitate methylation of DNA therein for more stable heterochromatinization.

With the acquirement of totipotency by the earliest embryo cells in association with occurrence of a chromatin structure that is the least condensed for somatic cells (22) and the separation of placental lineage from the embryo proper cells which have had repairs of the genome and repressions of retrotransposons to tolerable levels, the stage is set in the mammalian embryo for differentiations to the increasingly restricted stem cells whose further differentiated progeny will contribute to the formations of various tissues and organs. These cellular differentiations occur necessarily by heterochromatinizations of different regions of genome in different cell types. The heterochromatinizations, both constitutive and facultative, pose on the other hand constraints for repair and maintenance of genetic material and not only for the nucleic acid but also for the protein components and these are instrumental in upstream events of aging. The heterochromatinized sequences exist mostly at periphery of nucleus where tethers of DNA-bound proteins with particular elements of nuclear matrix-lamina-envelope associate them thereto and contribute to their repression. A subset of the heterochromatin associated proteins have been found to show low turnover or non-turnover and include particular nuclear pore complex proteins (33, 34). In proliferating S. cerevisiae (a unicellular eukaryote) these appear to be segregated to one of the progeny (“mother cell”) while the other receives the newly-synthesized counterparts and shows relatively longer replicative lifespan and the yeast cells in meiosis are found to specifically eliminate the oxidized and otherwise damaged proteins by their hydrolysis (33) while showing also suppression of mitochondrial oxidative metabolism and further means of protection from oxidative damage (35).

Multicellular eukaryotes are also determined to show likewise eliminations of oxidized and other damaged proteins during meiotic rejuvenation and to minimize production of reactive oxygen species by mitochondria besides promotion of a reduced redox state inside oocytes that contributes to their protection from oxidative damage (36, 37). Human oocytes show in this respect breakdown of the nuclear lamina-envelope as they proceed to the first meiotic division (the “germinal vesicle breakdown”) and the nuclear lamina-envelope of both maternal and paternal pronuclei are broken down completely prior to syngamy. Thus the initial cells of embryo forming upon the first embryonal cell division are set to de novo formations of heterochromatin with the associated elements of nuclear lamina-envelope-pore complexes by using newly synthesized proteins. The resting oocytes and particularly those nearing ovulation show enhanced reductive power and GSH/GSSG ratios many folds greater than even in the embryo somatic cells and appear to employ the reducing potential provided by GSH and other molecules for decondensation of sperm chromatin besides for maintenances of sulfhydryl groups of particular chromatin proteins of itself (37). These findings accord with the earlier likewise evidence and the conservation of basic mechanisms of meiotic rejuvenation from unicellular eukaryotes to mammalians (12).

Genome-wide distribution patterns of mutations in stem cells in various tissues of aging human (38) are also in accord with their origination from built-in conflicts in eukaryotes, in particular from those that heterochromatinization poses for repairs of oxidative and other types of damage as well as with their addressing during meiotic rejuvenation. Somatic mutations are found at increasing frequencies with increasing age of human in stem cells both in tissues where they show relatively high rates of proliferation (intestines) and little proliferation (liver) and these age-associated mutations are far more frequent in heterochromatin than in euchromatin (38). Mutations known to originate from failures and errors of repair of oxidative lesions and at CpG dinucleotide positions (where cytosine is commonly methylated and can undergo spontaneous and oxidant-triggered deaminations) were particularly enriched in heterochromatin (38) showing that the methylation of DNA, which evolved in eukaryotes in part for its utility against TE's, can also have costs in terms of aging.

Methylation of DNA and changes of it during aging, which include decreases and increases at different regions of genome in different cells and a decrease of the genome-wide average of 5mC in most tissues, may not however be taken as primary events of aging at least because the eukaryotic species that lack 5mC (e.g. S. cerevisiae, C. elegans, drosophila) also show aging with features like in other eukaryotes. Changes of methylation of DNA during aging appear to reflect rather consequences of and adaptive responses to the events further upstream in causation of aging. Thus the degrees of DNA demethylation in tissues during aging correlate with the degrees of DNA damage experienced (39, 40) and the nuclear lamina associated heterochromatinized regions of genome are found to be the predominant regions of occurrences of the DNA demethylations during aging (40) as well as of the age-associated somatic mutations (38) that can be caused by the failures and errors of repair of DNA damages therein.

The eukaryotes that survive and reproduce in anoxic and practically anoxic environments show energy metabolisms that do not employ oxygen and yet their genomes encode for proteins that are essential for meiosis across eukaryotes from anaerobs to human and they show multiple signs of meiosis (41, 42). The conservation and employments in anaerobic eukaryotes of a similar set proteins that are employed for meiosis also in human and other aerobic eukaryotes accord with the utility of meiosis for removal and repair of non-oxidative damages as well from the eukaryotic genetic material. In further accord, whereas oxidative damage beyond a threshold is found to cause entry of Schizosaccharomyces pombe to meiosis, this unicellular eukaryote, which can be facultatively anaerobic or aerobic, is found to enter to meiosis also under anaerobic conditions but at frequencies much less than under aerobic conditions (43).

### Difficulties of End Replication of Linear DNA do not Constitute Primary Causes of Aging

Telomeres, ends of the linear DNA molecules of eukaryotic chromosomes, show gradual shortening and increase of damage with increasing number of cell divisions and with increasing age in various tissues in human and other species. Number of cell divisions undergone by normal cells prior to becoming senescent correlates positively with the species-specific longevity among mammalians (44) and the proportion of senescent cells increases in tissues with increasing age of organism. Neoplastic cells, on the other hand, can undergo an unlimited number of cell divisions without telomere shortening and avoid senescence. These findings taken together with the knowledge that replication of the ends of linear DNA molecules poses difficulties and requires special measures, whereas the circular DNA genome of prokaryotes does not pose an end replication problem and the prokaryotes do not show a limitation of proliferation or aging, have led to assumptions of criticality of the end replication problem of linear DNA molecules in aging of eukaryotes. The end replication problem related to the linear DNA nature of eukaryotic genomes may not however be taken as a primary or upstream factor in causation of aging. Besides other reasons, experimental findings with S. pombe strains that have lost the catalytic subunit of telomerase and have lost the telomeric sequences but continued to survive as stable strains with circularizations of all chromosomes are relevant (45). These unicellular eukaryotes could enter to meiosis under conditions that promote aging and sporulation and they showed successful completion of meiosis despite the difficulties of untangling of circular chromosomes during cross-overs so as to achieve meiotic rejuvenation for their propagation for years (45). Further relevant are the findings where a naturally selected mechanism of sexual reproduction provides an internal experimental control. Females of mammals have two X chromosomes one of which undergoes facultative heterochromatinization during differentiation from the pluripotent embryonal stem cells for dosage compensation. The heterochromatinized X chromosome resides in the same cell nucleus with the active X during lifetime of women. Chromosomes in the lymphocytes obtained from newborn girls were found to have similarly long telomeres in both X chromosomes and in autosomes and then showed gradual shortening with increasing age of women in both X chromosomes as well as in autosomes but to significantly greater degrees in telomeres of the heterochromatinized X (inactive/active X chromosome telomere length ratios of ˜0.97 in newborns, ˜0.71 in 29-40 years old women, ˜0.55 in 60-70 years old women) (46). The active and heterochromatinized X chromosomes' telomeres are served by the telomerase and factors supplied by the same cell in the same nucleus, have proceeded through an equal number of cell proliferations and would have been exposed to likewise amounts of oxidants and other damaging agents during the lifetime of a woman but have one critical difference that is in the structure of chromatin.

Also, in comparison to the active X residing in the same cells of aging women, the heterochromatinized X is found to show significantly greater frequencies of repair failures and somatic mutations at its nontelomeric sequences as well (47). Thus the structure of chromatin and its modifications for heterochromatinization that originated in unicellular eukaryotes and were employed during evolution of multicellular eukaryotes, while necessary for the generations of different cell types having different phenotypes and functions in human, are at the roots of biological aging and the repair failures and shortening observed at telomeres with increasing age of organism are part of the consequences.

### Looping of Nuclear DNA and Modifications of Chromatin Employed in Regulation of Cellular Differentiation Pose Constraints in Maintenance of Genome Integrity Bypassed in Neoplasia

Previous investigations revealed that the nuclear lamina-matrix proteins that remain associated with nuclear DNA at high ionic strengths that dissociate the histones and most of nonhistones include a protein of ˜220 kD apparent molecular mass by SDS-PAGE that in normal cells shows intermolecular disulfide bonding with a peptide or protein that is covalently bound to DNA (12, 15). This subpoulation of ˜220 kD protein consistently showed increases in quantity with increasing age in human and mouse normal tissue cells whereas neoplastic cells did not show detectable intermolecular disulfide bonding of the ˜220 kD protein and, in comparison to their normal counterparts, had also lower quantities of it in intact nuclear DNA-lamina-matrix complexes purified by ultracentrifugation through neutral sucrose density gradients (12, 15). The evidence for peptide/protein species bound to DNA by covalent or covalent-like bonds and with which a subpopulation of the ˜220 kD protein involved in the folding of nuclear DNA to large loops showed disulfide bonding in normal cells included the former's resistance to dissociation from DNA by 1.2% or more SDS at 100° C. for 10 minutes or longer (12, 15) and causation of disappearance of such molecules from DNA by proteinase K (48). Further in accord, physical shearing of DNA of the purified intact nuclear DNA-lamina-matrix complexes of normal cells prior to nonreducing SDS-PAGE revealed mostly diffuse banding by the ˜220 kD protein contrary to the sharp bands of the other proteins resolved in the same gels and showed in addition DNA fragments displaying smeared staining starting immediately below the ˜220 kD protein and continuing downwards with decreasing intensity when visualized by a method that detects both protein and DNA components at the same time and the treatments of complexes with disulfide reducing agents and/or with DNAse I prior to SDS-PAGE caused sharpened bands of the ˜220 kD protein simultaneously with the disappearances of DNA fragments (12, 15, 49). Since DNA shows markedly increased electrophoretic mobility in the presence of SDS (49) the physically sheared DNA fragments remaining bound to the ˜220 kD protein during nonreducing SDS-PAGE apparently elute off the gels under the reducing SDS-PAGE conditions resolving the ˜220 kD protein (12, 15, 49).

Normal cells showed in addition intermolecularly disulfide bonded larger species of the ˜220 kD protein that resisted entry to 4% polyacrylamide gels under nonreducing conditions and this fraction showed significant increases with increase of age in human and mouse (12, 15). The ˜220 kD protein appears to participate in the folding of nuclear DNA to loops in human and other mammalian cells, predominantly in the 60-110 kb range (48), and normal tissue cells appear to have age-associated increases of its oxidatively modified forms and of a subpopulation that shows disulfide bonding to a peptide or protein that is covalently bound to DNA (12, 15). The following findings point to the occurrence of the oxidative modifications of this protein and of a subset of other nuclear lamina-matrix proteins in vivo during aging: (i) purification of the intact nuclear DNA-lamina-matrix complexes by lysis of live cells on top of density gradients in buffers preventing artefactual sulfhydryl oxidation and sulfhydryl-disulfide exchange reactions (12, 18), (ii) further ruling out of such artefact by the brief cell lyses in cold prior to the centrifugal separations of complexes (12, 18) and (iii) highly reproducible effects of the reduction of disulfide bonds of the DNA-protein complexes on their conformation, sedimentation rate, morphology and light scattering characteristics (6, 12, 18, 19). The consistent nondetectability in neoplastic cells of the ˜220 kD protein subpopulation found to show disulfide bonding in normal cells to a peptide/protein covalently bound to DNA relates accordingly to the escape of neoplastic cells from senescence.

What may be the covalently DNA bound peptide/protein species revealed in the earlier investigations to exist in disulfide bonding with a subpopulation of nuclear proteins in normal tissue cells that showed increase of such during aging (12, 15)? Unlike expected for covalent protein-DNA crosslinks generated randomly, earlier experimental findings accorded rather with nonrandom positions of the detected disulfide (S—S) bonded proteins along nuclear DNA, at or adjacent to the bases of DNA loops, in view of the unfolding of loops to larger ones upon reduction of S—S bonds (12, 18, 19) and release of predominantly 60-110 kb DNA fragments upon S—S reduction (but not without) when limited nonrandom restriction endonucleolytic cuts were introduced to the intact nuclear DNA found to be folded to supercoiled loops associated with nuclear lamina-matrix (48). Other experimental approaches to assessments of the supercoiled DNA loops included their detections in situ in individual cell nuclei visualized under microscope with and without treatment with disulfide reducing agents (19). Results of those investigations also accorded with occurrences of disulfide bonds at bases of chromatin loops and suggested loop sizes in a few hundred kb range considering the dose-response curves of γ-ray induced DNA strand breaks (19). Regulators of supercoiling of nuclear DNA determined since then have shown the eukaryotic DNA topoisomerases as major players with significant presence at bases of DNA loops in interaction with particular chromatin proteins that include the CTCF protein that recognizes the CTCF target sequences repeated throughout genome in human and contributes to the folding of nuclear DNA to loops (reference 50 and references therein). Both type I and II topoisomerases are in this respect known to covalently bind to DNA transiently for topoisomerase function and to form stabilized covalent enzyme-DNA complexes when enzyme fails during catalysis. Instructively, the molecular pathways for their removal and for repair of the resultant DNA lesions are conserved from unicellular eukaryotes to human and found to be causes of accelerated aging when compromised by loss-of-function mutations (51). Differential bindings of CTCF and of other DNA looping proteins to their target sequences are employed for regulation of chromatin structure of the looped domains and of their positions in nucleus and they can affect repair of DNA also by a strategic positioning of topoisomerase II at anchor of loops (50). The nondetectability in 5 neoplastic cells of the subpopulation of the ˜220 kD protein found to show S—S bonding to a peptide/protein covalently bound to DNA in normal tissue cells at increased quantities during aging (12, 15) may accordingly relate to the defective maintenance of integrity of genome in neoplastic cells.

Limitations Posed by Human Brain to Interventions with Aging of Human May not be Absolute

Human brain, the site of human intelligence, poses further challenges to intervention with aging. Not only the critical mechanisms of aging pointed above are operative also in the central nervous system (CNS) but the generic functions like learning and the uniquely human CNS functions (e.g. abstract thinking) depend on the modifications of chromatin in the cells of neuronal circuits that are formed, modified and employed for them. On top of those established during evolution of human brain, constraints occur in interventions with aging of brain arise also due to the fact that particular neuronal circuits and cell-tissue structures operating in each person and providing his/her memories and intellectual functions have formed through past events starting with intrauterine development. In testimony of the constraints established during evolution, chromatin remodeling events in cells of neuronal circuits that may be targeted by pharmacologic, molecular genetic and cell-tissue engineering means are sensitive to even a single component's ˜50% change of amount as shown by the intellectual disabilities and psychiatric problems caused by particular remodeler haploinsufficiencies (52). Reliance on terminally differentiated neurons for adult CNS functions adds to the difficulties of upkeeping of CNS functions at ages beyond the average human lifespan. Although neurons are endowed with means of minimization and repair of damages to the genetic material, the differentiation from stem cells to progenitors and then to terminally differentiated neurons imposes restrictions to repairability of damages and to the replacements of chromatin constituents similar to that in other terminally differentiated cells (34, 53, 54). In addition the chromatin structure in the multipotent stem cells that are capable of supplying new neurons in the CNS of adults (53) do not have the chromatin structure advantages that the embryonal stem cells and their precursors have (22, 30). Analyses of human CNS confirm that the heterochromatinized regions of genome in neurons are particularly susceptible to failures of repair of the damages to genetic material and provide evidence that such failures are major contributors to the age-related CNS functional decline and neurodegeneration (54-56). On the other hand, whereas the age-associated losses of CNS functionality are among the highest of the costs of aging on patients and on society without a solution in place today (1-3), basic upstream mechanisms of aging are likewise in the CNS and elsewhere in human body and effective interventions with aging of CNS can be performed as I point below.

### Shortcomings of Forced Expressions of Pluripotency Conferring Transcription Factors in Somatic Cells

Comparisons of gene expression profiles of pluripotent stem cells with their restricted and further differentiated progeny have revealed pluripotency-conferring gene products and investigations of forced expressions of them in somatic cells where their expressions had been downregulated confirmed that the constraints imposed by the modifications of chromatin for cellular differentiation are critical in aging of organism. Thus, forced expression of pluripotency-conferring proteins in somatic cells of adults is found to cause senescence or apoptosis of high proportions of them and a subpopulation of the cells resisting senescence and apoptosis is found to produce tumors (57). Causations of in situ neoplasias are found with forced expressions of pluripotency factors for short durations and undifferentiated invasive-metastasizing tumors are found with longer expressions (57). Furthermore, with increasing age of animals increasing proportions of the somatic cells are found to undergo senescence or apoptosis upon forced expressions of pluripotency conferring proteins in them for production of “induced pluripotent stem cells” (iPSC's) from them (58) and the iPSC's generated without corrections of the somatic mutations acquired by somatic cells during aging carry high risks of giving rise to tumors (57, 58). Such iPSC's would not provide a solution to neoplastic or other diseases of aging. Subjecting iPSC's to the repair and further processes that epiblast-derived germline cells go through, followed by in vitro fertilization of derived oocytes and transferring of the obtained embryos to pseudopregnant females, have on the other hand shown productions of apparently normal males and females, albeit at low success rates, that were fertile (59).

### Treatments of Age-Associated Diseases

Diseases of aging without a satisfactory treatment are frequent and have been investigated worldwide for developing treatments. Conventional approaches to development of a new drug treatment employ screening of libraries of molecules with in vitro assays hypothesized to be useful, proceed with positives to in vivo tests using simple laboratory animals and, in case of positive(s), tests are carried out with higher species and, in case of positives with them, further tests are carried out in clinical investigations. A new drug treatment considered to have shown acceptable therapeutic efficacy in the clinical investigations is then submitted to a regulatory authority for approval. This is a lengthy process and reported to have been providing increasingly diminished returns despite costs at historic highs at present.

The fact that diseases of aging occur through complex processes and typically affect many molecular events, tissues, organs and systems that create astronomical numbers of a priori possibilities lowers the likelihood of success by the conventional approach to development of new drug treatments. Accurately determining decisive upstream mechanisms of pathogenesis of a complex age related disease is in general a precondition of development of an effective treatment as an upstream event can have numerous consequences in the affected patients upon which diagnoses of disease are in general made.

Cancer is strongly associated with aging. Among the treatments for cancer, surgical excision has been widely practiced and can provide cure when properly done but it can cause losses of organs and functions of patients and turns out unfeasible for a large proportion of patients due to an unsuitable location or late stage of disease that preclude surgical excision for cure or a benefit to patient. Conventional chemotherapy-radiotherapy of cancer have in general been used for these patients. Experience with them shows that whereas some patients may be cured, majority ends up being killed by cancer even when some initial response (slowing of growth, decrease of size or undetectability of tumor) is observed, often with relapsed or persistent disease unresponsive to further treatments. Patients undergoing conventional chemotherapy-radiotherapy commonly experience serious unwanted effects arising from harming of normal cells and such may include causation of death of a patient by the treatment. These nonsurgical treatments of cancer commonly act by causation of damage to the genetic material and can cause death of tumor cells when excessive.

Taking into account (i) causations of mutations and cancer by genotoxic agents, (ii) occurrences of somatic mutations and of cancer during aging, (iii) increased frequencies of unrepaired/misrepaired damages in somatic cells during aging, (iv) effects of structure of chromatin on outcomes of damaging of genetic material, (v) dependence of cellular differentiation on formation of heterochromatin at particular regions of genome, (vi) prevention of cellular differentiation in increased proportions of cells in tumors relative to the cells in corresponding normal tissues, (vii) occurrence of senescence in normal somatic cells and its causal relation to aging of organisms, (viii) frequent escape of cancer cells from senescence, and (ix) further observations about aging and tumorigenesis discussed elsewhere (12), my investigations have been focused on mechanisms of aging and of tumorigenesis during aging and on their relations with structure of chromatin. Cells in normal tissues occur at varying states of differentiation from stem cells to the terminally differentiated at spatially distinct positions in relation to the cells of own lineage and to cells of other lineages that facilitate specific interactions by secreted molecules for regulation of differentiation. The terminally differentiated cells typically show more facultative heterochromatin and damage in genetic material than in other cells and, when damage is beyond a threshold, are eliminated by programmed cell death for replacement by proliferation and differentiation of precursors. In this hierarchy the stem cells have a top position, occur at special tissue positions (niches) where they are supported by other cells and are maintained as the least differentiated cells of their lineage which helps their long term survival with relatively low damage in genetic material to serve as sources of differentiating progeny during lifetime of human. They proliferate rarely unless required for tissue homeostasis and can divide asymmetrically to give rise to a stem cell and to a differentiation-committed cell which typically is expelled from the niche. Despite being privileged, stem cells also show increase of mutations and misrepaired/unrepaired damages with increase of age. Our investigations have shown that tumor cells show, in comparison to their normal counterparts, consistent modifications of structure of chromatin and of nucleo-cytoskeleton that relate to their escape from senescence and to their survival and proliferation as undifferentiated cells at tissue positions away from their first occurrence, indicating that the mutations and epigenetic modifications they have acquired provide the tumorigenic cells independence of an anatomically defined niche for self-renewal (references 6, 12, 15, 19, 60 and references therein). These advantages of tumor cells over normal cells in untreated patients are turned to their disadvantage upon a medicament administration designed to target the identified differences of tumorigenic cells from normal cells (6, 60). Investigations with tumor bearing human have shown provisions of beneficial therapeutic results undescribed with and not inherent in the previous treatments of tumor bearing human (60). These beneficial therapeutic results include the rapid disappearances of tumors without relapse irrespective of histopathological class of tumor and desirable safety findings (60).

Determinations of mechanisms of an age-associated disorder at various levels may on the other hand not suffice for effective intervention when a critical event of pathogenesis is not accurately understood or an intended intervention turns out impossible due to unwanted effects on important physiological functions. Unavailability of an effective intervention in market may be due in today's world also to nonscientific-nonmedical reasons that can preclude a scientist having objective evidence of an effective new treatment that has shown desirable safety in human (see below). An example for a targetable common health problem is the age-associated decline in the amplitude of accommodation of human eye which, from an economics perspective, represents a major market worldwide. Which age-associated changes of the eye lens bring decline of accommodative power, the oxidative modifications of lens proteins causing their cross-linking/aggregations that interfere with light passage, contributions by nonenzymatic glycations of lens proteins and even eye lens changes that correlate with species-specific MLP have been described (61-63). Yet, except for the wearing of synthetic lenses or cornea/lens surgeries having limitations and risks, satisfactory intervention with the age related disorders of eye lens have not been available and clinical testing of treatments thought to target upstream events of pathogenesis have so far shown marginal or no significant benefits (64).

### Underutilized Worldwide Potential for Advancements in Science and Technology: Evidence and Relevance

Advancements in science and technology have been essential for advancements of humanity. These are usually made by incremental additions to the existing knowledge but occasionally a breakthrough in a field of science and technology, typically by someone who has departed from the prevailing assumptions, opens up new ones and may render the preexisting technologies obsolete. Considering that genuine scientific-technological advancements have been decisive in enablements of human beings but most of them have been brought in relatively few industrialized countries of world, a serious unsolved problem can be recognized when taken together with the facts of human biology. The earlier industrialized countries providing most of the contributions to science and technology (65) contain a small proportion of world's population and skewing in the distribution of the contributions by world's countries to science and technology becomes worse with normalization of the data for per capita contributions. Considered with the basic facts of human biology and genetics that have been pointed earlier, this skewing indicates a vastly underutilized worldwide potential for achievements of scientific-technological progress for advancements of humanity. People in all countries are heterogeneous to result in a spectrum of capabilities in different spheres of life and in accord improvements in allocation of resources to scientific-technological research on basis of merits along with higher education is found to provide marked improvements of the scientific-technological competence in a country where such had been poor (66).

Settled scientific-technical knowledge disseminated by scholarly books has been serving well the students of science and newcomers to a field. Dissemination of new findings in science and technology has on the other hand not been as smooth. Quality control of a communication purporting new findings in science helps to filter out the mistaken and useless by “peer review” and contributes to progress of science when it is done by accurate objective assessments of a described scientific matter. Yet betrayals of trust, deliberate blockade of the worthy for self-benefit, reviewer incompetences and cheating have all been known and observed also in reviews of applications to public research funds (66-68). Although facts can be sorted in the long run, significant losses of time and resources due to such flaws remain as a problem. A means of amelioration in that respect is pointed below. Achievements of slowing of rate of aging of human and effective treatments of major age-associated diseases versus remaining at a scientific-technological level where the rate of aging remains the same as before and the treatments of leading diseases of aging continue to be symptomatic necessarily have social and economic consequences and they are eventually worldwide as also pointed below.

### Effective Addressing of the Aging Associated Problems in Human Societies Demand Social, International, Economical Overhauls Besides the Targeting of Basic Biology

Problems caused by the age-associated disorders to individuals and to society cannot be effectively solved by the symptomatic treatments of these disorders although patients may be helped by alleviations of symptoms. Population growths around the world that lower frequencies of older age groups and thereby the frequencies of age-associated disorders have been a means of avoiding the problems at levels of human societies without an actual solution for the basic problem. Population expansions leaving the basic problem unsolved are on the other hand unfeasible to sustain in the long term in a planet having limited size and resources and where effects of such are no longer containable within national borders and where absence of effective solutions for the basic problem have increasing costs (1-3). Hence interventions that slow the rate of biological aging in human, methods of repairing/reversing the effects of aging and developments of effective treatments of the common age-associated diseases appear to be the only viable and humane options for the long term.

Practices of economy that are reliant on expansion of human population for economic development therefore require reassessments for solutions of the problems arising from aging. In the absence of effective treatments of major age-associated disorders, the economy practices that depend on continuous population growths for economic development and growth face predictably increasing social and economic costs arising from the increasing frequencies of these disorders and disabilities (1-3) and a planet of limited size and resources is unsuited to sustaining of continuous population growths. Effective treatments of common age-associated diseases and slowing down of aging of human would start to provide benefits and help to counter the problem but realizing and implementing them appear to have requirements that are poorly fulfilled in today's world as revealed by the new drug treatment case described in Example 3.

Because effective interventions with aging and age-associated diseases of human have consequences beyond the medical and because the invention and findings presented herein have revealed particular shortcomings in those non-medical areas, the presentation herein includes descriptions of them to the extent that they pertain to the primary field of the invention. The descriptions inform in addition about improvements in nonmedical areas that can facilitate implementations of the biological-medical solutions

Industrial societies where mass productions for mass markets have been a driver of the economies and associated social stratifications have emerged relatively recently in the history of human species, within a few hundred years from present. A few hundred years, while sufficing for the rapid increase of world's population and creations of the social-demographic-medical problems experienced today, might not have been sufficient for fact-based determinations of social, economic, international systems optimal for the human species. Iyengard & Massey (69) describe in this regard that the social infrastructure and institutions coming from past have been unprepared to the rapid developments in the electronic and communication technologies that have created marked changes in social and economic spheres within a few decades that include the unforeseen and some not positive and it is pointed out that shortcomings of human CNS create much room to diversity in social dynamics (70). Reminding that new technologies can facilitate further scientific-technological progress but may also be used for illegitimate purposes, uses of electronic monitoring, through-walls-radar and related technologies for intruding private communications and activities of individuals and automatable internet-based spread of tailored disinformation to individuals and masses for monetary and political gains have been described along with their negative effects on social fabric. These can be particularly detrimental for the growing unsolved problems arising from aging in human societies since science can only be built on truth and verifiable facts whereas conditions permitting or even favoring deceptions and theft (69) would undermine the progress to actual solutions. Further relevant are the facts that internet, electronic communication devices and electronic social media are in general black boxes for the majority of users in terms of the technologies behind (which can be enabled, disabled or modulated selectively by the few controllers and technical experts having the necessary resources) and the relative ease of exploitations of people by their means (69) would not make their proceeds suited for solutions of the problems for which large sums have been spent and the conventional approaches have not produced results beyond palliation (1-3, 11). Considering that carrying out of a scientific-technological project by a person or group requires an essential level of economic resources but solution of a significant scientific-technological problem is not guaranteed by any amount of money, a globally applicable improvement is pointed below based on the patent system and improvements thereto.

The patent system is a means of promotion of progress of science and technology that sidesteps the existing difficulties in merit-based assessment and support of research proposals since a patent is issued normally to someone who already has demonstrated solution of a scientific-technological problem where the solution is unobvious and industrially applicable. It confers limited rights to a patentee and has been found to stimulate scientific-technological research and progress in the countries where it is introduced. A patent is supposed to be issued only to someone who has shown factual evidence of a novel and unobvious technological solution and therefore proper functioning of patent system is critically dependent on the quality of examination of applications. In view of the increasingly global trading and need for international cooperation, the Patent Cooperation Treaty (PCT) has been made that allows filing of an international patent application and description of a new technology in one signatory state with effective date in all signatory states. PCT has not however progressed to a world patent system that, once realized, can provide improvements in utilization of the worldwide potential for advancements in science and technology. An immediate reason is that an application with effective date for all signatory states must still be separately filed in them with all the costs and formalities involved and, more importantly, the decision of whether an operative, novel and unobvious technology has been shown in the specification of application is left to each signatory state without a safeguard in the treaty for a decision on basis of merits. Neither an assurance exists that a signatory state would have the necessary infrastructure and qualified examiners in every field of technology to be able to examine properly nor a safeguard exists that a government that recognizes that a scientific-technological breakthrough made in another country and predicted to be disruptive to major industrial activities and strengths in its territories would remain impartial in examination and patenting of that technology in its territories.

An international patent authority with power to examine international applications for a patent effective in all signatory states (ideally, and probably eventually, in all of world) would eliminate the problems and conflicts inherent in examinations of international applications by national offices. Such an international authority, staffed with manager and examiners from around world on basis of merits, would have greater capabilities not feasible for a national office. Whether a purported technological advancement is true and previously unknown can certainly be objectively determined by those skilled in its field and there are known criteria for reasonably objective determination of unobviousness. An international authority empowered and mandated to decide about international patent applications objectively according to merits would avoid the conflicts inherent in national patent offices and reduce the quality concerns, costs and inefficiencies arising from separate examinations in individual national offices that typically have varying capabilities, formalities and accountabilities. It would serve for efficient utilization of the worldwide potential for progress of science and technology. Present utilizations of that potential, e.g. by attracting scientists from poor countries, might have had positive effects but have inadequacy and problems. An individual born in a poor country and having advantageous innate features for intellectual achievements may fail to express it even for mundane reasons and due to poor research facilities considering that such countries tend to have poor infrastructures and low likelihoods of making them available on basis of merits (71). A scientist in an underdeveloped part of world, when he or she solves an important scientific-technological problem despite the suboptimal conditions, would be able to present the findings to such an international patent authority for proper objective examination. An authority allowing patents solely on basis of merit and probably providing also support for their bringing to market without the need for a describing scientist to spend his/her time for matters for which he/she does not have expertise would enable a scientist born anywhere in the world in acquirement of resources to continue to work for further advancements.

European Patent Office (EPO) shows in this respect that a single international body can be functional in examinations of applications for patents in numerous countries with capabilities far greater than it had been possible in individual national offices. Whereas EPO may need improvements, establishment of a global authority for examinations of patent applications from around the world appears to be able to bring decisive improvements to utilization of the worldwide potential for scientific-technological progress.

Recourse to Facts of Human Biology in Global Measures Against the Problems Arising from Aging of Human

The hitherto unsolved problems originating from aging of human affect all people irrespective of national identities and the extents of these problems in human societies have been increasing across the world. Measures at global level would be best for such problems. Yet shortcoming and failures in achievements of global level actions have been common. Analysis of the main causes and means of overcoming of them would thus be appropriate.

Failures of appropriate international action where such is needed may in part have origins in the susceptibility of large proportions of people to being led by unfounded assumptions and emotions (70). Since emotions can be instrumental in social cohesion, they are commonly relied upon by politicians (69) and it is generally easier to divide people along national lines than bringing together for transnational-global concerns. However none of the defining features of nations is a constant when analyzed over periods spanning several thousand years and the sequencing of genomes of people from different continents and nations (10) show their common origins and lack of an essential reason or basis for divisions of people along national lines. The term nation refers to people sharing a language and history in a particular territory which in turn leads to shared culture. Lack of necessity of divisions of people along national lines is evidenced further by considering that language and culture, definers of national identity, are learned traits and cooperation of nations towards solutions of common problems would not require abandonments of the values built by ancestors of people in nations.

A further divide hindering cooperations across world is observed to relate to differences in religious affiliations. Religion is a strong effector of emotions and ideals of large proportions of people and has also been described to be a significant effector of economic outputs of countries (71, 72). Explanations vary for the correlations of religious affiliations with per capita economic outputs and contributions to science but the strength of separation of church and state is said to be a significant factor (71, 72). Religion is recognized to be an effector of social cohesion and culture of nations and a facilitator of governmental operations. When considered from an international perspective, however, reliance on religion for political ends may require taking into account the facts that (i) major religions have ancient codes and a politician intending to rely on religion for a matter is constrained by them, (ii) worldviews affected by a religion in a human population may have little receptivity in another and may even be at discord, and (iii) some religions teach others as being inferior. Also some worldviews and religions perceive the world as being driven by struggles of positive and negative where clashes of the two help to bring out truth and selection of the fittest. However under the social and international conditions existing today and with availabilities of satellite networks, electronics and further technologies enabling targeting of individuals and groups of people from a remote location for harming, the selection conditions may favor those fit to undermine progress of humankind and of science as well (69). Considering an international system not so prepared to these conditions, inadequately assessed relying on religion may accordingly risk entering of politicians and governments into positions that they may find difficult to correct for globally coordinated actions required for solutions of global problems.

Affiliations of world population with a particular religion or no religion at frequencies ranging from ˜15 to 30% for the common and ranging from less than 10% to less than 1% for the other religions (73) do not therefore create hindrance of globally coordinated actions demanded by global problems when leaders of political groups and governments avoid basing of policies on a religion in manners that can bring them into a position from which they cannot exit (or when those failing are set aside). The reality that affiliations with particular religions or no religion are also learned traits and that majority of people innately value justice in social relations irrespective of their religious affiliation (74, 75) further indicate that there is not an insurmountable blockade in front of worldwide unified actions by the existing differences of religious affiliations of people.

Establishments of existing major states through past wars that could have left prejudices and conflicts may also be a factor hindering international cooperations for solutions of global problems. On top of it, unpreparedness of the social and international institutions of past to the recent technologies used for affecting behavior of masses of people for illegitimate gains are recognized to have negative social effects (69) and the social environment in countries can affect their international relations. Recourse to the facts of human biology can help also in this respect. Whereas social science has become increasingly biological science through insights into mechanisms of functioning of human CNS, adjustments in social and international institutions, including the law, and not only within nations but ultimately globally, appear to be the most feasible for solutions in a timely manner at present and they would also pave for subsequent improvements. The fact that solutions of complex multicomponent problems do not occur without solution of every component and yet blockade of any component suffices for a party perceiving benefits from delays may also be increasingly relevant under the social and international conditions today and national-international legal provisions for retrospective accounting in such cases would accordingly be useful. The findings that people around the world seek justice in social systems and relations irrespective of their national origin and religious affiliations (74, 75) imply roots in basic human biology. Its upholding can thus be a principle also in in international relations in solutions of complex problems that include those poised to worsen worldwide as a result of the increases of average human lifespan in the absence of effective treatments of disorders of aging.

### Key Processes Affecting Rate of Aging and MLP of Human and Tailored Modifications of Genome and Metabolic Processes in Human Cells

Biological aging results from inherent conflicts and shortcomings in eukaryotic organisms (12) that could not have been eliminated by natural selection during the about 2 billion years since the first occurrence of eukaryotic organisms and moreover the easier to achieve modifications have already taken place in the human genome while providing the present MLP of human. Yet there is not a basis for illusion of invincibility of aging at least because identifications of the decisive upstream causes of aging point to means of effective interventions in manners not possible by natural selection as described in this invention.

Analyses of the data with non-human species in which significant slowing of rate of aging and increases of MLP have been caused suggest limited applicability of them in human. For example investigations of restrictions of calories in diet of laboratory animals indicate generally greater degrees of increase of maximum lifespan in species having relatively shorter MLP (e.g. references 7-9). It may imply potential of relatively little increase of MLP by caloric restriction in human having about 30 fold or longer MLP in comparison to mouse and far longer MLP than in the studied primates. Laboratory animals allowed to eat as they wish may be consuming generally more than available in the wild and may accordingly be viewed to show acceleration of aging relative to the restricted animals but the conclusion remains the same: maximum lifespan is extended in diverse species by the caloric restrictions attainable in a species whether in the wild or in laboratory and the extensions of lifespan by caloric restriction relate inversely to the MLP of species. Analyses of the animals subjected to caloric restriction show also a common effect in all investigated species: causation of decrease of oxidative damages in genetic material in cells of restricted animals in comparison to those unrestricted. Lowering of oxidative damages in other cellular constituents such as in various membrane lipids have also been found with caloric restriction. However, unlike the critical damages in genetic material, the damages in these other cellular constituents can in general be remedied without irreversible consequences.

Aforementioned and earlier presented determinations accord with lowering of the oxidative damaging of genetic material for increases of the species-specific MLP by naturally selected changes in genomes of eukaryotic species during their evolution. It is also clear that the optima in amino acid sequences of proteins, in gene combinations, in the genome sequences participating in regulations of expressions of genes and in the metabolic pathways that they affect and have been provided by the ˜2 billion years of natural selection in the occurrence of present day human genome and MLP do not suffice for preventions of the problems accompanying aging of human. Human is on the other hand no longer limited by the natural selection-mediated modifications of genome. Advancements in molecular genetics have provided methods for insertion, deletion or changes of nucleotides at desired positions of a genome and they can be used for tailored modifications of metabolic and further processes in cells and at whole organism level for causation of decrease of rate of aging and increase of MLP in human as described herein. Genome sequence determinations have shown the nucleotide sequences of human genome both in terms of those common among individuals and those polymorphic at particular positions (www.ensembl.org/Homo_sapiens and www.ncbi.nlm.nih.gov/grc/human are among the resources of such information via internet and prints of sequences and data in other media have also been available). Methods for DNA sequence determinations and synthesis of DNA molecules of a desired sequence and length have been known.

Modifications of human genome sequence can be performed in part in test tubes with DNA fragments corresponding to an intended segment of the genome and a modified DNA fragment ligated to a plasmid or viral vector DNA can be introduced to human cells in tissue culture for incorporation to human genome. Methods for excision of a sequence from genome of human cells are also known. CRISPR/Cas (Clustered Regularly Interspaced Short Palindromic Repeats (CRISPR); CRISPR-associated protein9 (Cas9)) methodology employs guide RNA molecules for targeting an engineered Cas9 protein to a target position of genome in cells for introduction of strand breaks to DNA therein for changing the sequence therein. An example of use of the CRISPR/Cas methodology for excision of desired segments of the genome from human cells is described in reference 76 which describes use of it for removal of transplantation antigens from human cells. Since the CRISPR/Cas methodology relies upon causation of double strand (ds) breaks to DNA, which can be repaired by homologous recombination (HR) in cells, and since HR can introduce unwanted deletions or insertions of nucleotides to cause unwanted mutations and since causation of dsDNA breaks can have other undesired consequences as well in the affected cells, safer and more accurate methods of modification of genome sequences in human cells have been investigated. Reference 77 describes use of an engineered Cas9 protein fused with a reverse transcriptase (RT) protein modified from the MMLV RT for modifications of sequence of human genome without introducing dsDNA breaks to the genome. Targeting of the fusion protein to desired positions of genome by use of guide RNA molecules is described and particular versions of the method are described wherein cells showing the intended nucleotide changes at target positions include those lacking a detectable unintended deletion or insertion (77).

Enzymes acting on nucleic acid molecules in prokaryotes and eukaryotes contribute to the tools used in molecular genetics. Reference 78 describes engineering of an E. coli TadA protein for use in a method that enables change of an adenine (A) nucleotide at a desired position of genome to a guanine (G) nucleotide. It refers to an earlier described such method and to its shortcomings due to causations of deaminations of A also in the RNA molecules in the transcriptome of cells and describes substantial decreasing of A deamination of RNA while retaining the A deaminating activity on DNA. Similar methods using Cas9 fused with other engineered deaminases for deamination of cytidine (C) for changing C at a desired site of genome are also known. Spontaneous and oxidative stress-triggered deamination of 5-methylcytosine (5mC) that converts it to T and creates thereby a mismatch in the dsDNA (T: G mismatch in place of the original 5mC:G match) is a risk of occurrence of mutations in the affected regions of genome. Mutations can occur when the DNA is replicated before repair of the mismatch and also due to the errors and failures of mismatch repair of DNA in the heterochromatinized regions of genome. The heterochromatinized regions of genome are burdened by this type of DNA damage additionally because the 5mC modification of C contributes to the heterochromatinization. The 5mC; G matched site converted to T: G mismatch can thus be converted to a T: A match which however is a mutation. In this regard C to T transitions are the most frequent type of mutations detected in organisms employing oxygen for energy metabolism. Accordingly the Cas9 fusion proteins referred above (and other similar fusion proteins) can be used for countering effects of aging besides the more effective upstream interventions with aging described below. Uses of engineered Cas9-adenine deaminase and Cas9-cytidine deaminase fusion proteins for making desired nucleotide changes in genome have been described in many species. Reference 79 describes uses in human cells to change nucleotide sequence at a desired position in repetitive sequences and describes stably maintained human cell clones in which an intended nucleotide change was caused in tens of thousands of copies of a repeated sequence.

Not all nucleotide sequences existing in genomes of eukaryotes provide a function useful for the organism and some of the sequences and genes that might have had useful effects in the past and under different environmental conditions may not have a useful or essential function today. Screening methods used with unicellular eukaryotes to determine which genes in their genome may not be essential for their survival and reproduction have shown dispensabilities of more than 20% of the genes individually in ordinary cultures and have also shown that more than 25% of the genes found to provide an essential function in the wild type organism became nonessential upon causations of mutations of other genes of the organism (80). Constructions of synthetic chromosomes wherein nonessential nucleotide sequences existing in the wild type chromosomes are removed have also been described along with the features of the unicellular eukaryotes in which a wild type chromosome is replaced by an engineered chromosome (e.g. ref. 81). Constructions of synthetic chromosomes with symmetrical loxP sites downstream of genes and subjecting a unicellular eukaryote having such chromosomes to a conditionally activatable Cre recombinase to cause recombinations and deletions of multiple genes to determine the particular subsets whose simultaneous deletions and inactivations would not undermine the survival and reproduction of the organism have also been described. In case of the Saccharomyces cerevisiae chromosome XII left arm, deletion of more than half of the sequences therein has been described to be compatible with continued survival and reproduction of the organism (81). Subjecting unicellular eukaryotes to evolution by causations of recombinations and deletions as referred above and by other such methods that are also known in the field are in principle extendable to cells of multicellular eukaryotes in culture. Doing so with e.g. embryonal stem cells (ESC's) can provide cells that have survived desired particular in vitro evolution conditions which then can be subjected to in vitro screening tests to discard those that have survived during the in vitro evolution but determined by the in vitro screening not to be fit for functioning in vivo before testing the remaining cells for functionality in tissues of animals. Methods that enable incorporations of cells to e.g. monkey embryos for follow up of their functioning in the animals have been known.

Above referred and described methods of causations of desired nucleotide sequence changes in genome of human cells can be used for tailored modifications of metabolic and further processes in human cells for causation of decrease of rate of aging of human and for effective interventions with disorders accompanying aging. A basic task exists on the other hand for those skilled in the field and intending to proceed: determinations of the particular nucleotides and genes to be targeted out of the astronomical numbers of potential combinations. Existence of more than 3 billion nucleotides in the haploid human genome corresponds in the absence of a guide to more than 3 billion candidates individually and their combinations rapidly become astronomical.

With determinations of the upstream mechanisms and molecular events that are decisive in causation of aging of human, presented in this invention, the particular modifications that can be caused in the human genome sequence for particular modifications of the described molecular events and processes for interventions with aging and diseases of aging of human become evident to scientists skilled in the areas of present invention. Exemplifications and additional directions are also presented below for further guidance in practice of the invention.

The following summary of the findings serves for distinguishing of the upstream mechanisms and primary causes of aging from the consequences downstream for targeting of the former for effective interventions with aging and disorders accompanying aging in human.
(i) Structure of the eukaryotic genetic material that constrains repair of damages to it is a primary cause of aging. Heterochromatinization of chromatin at particular regions of genome that is essential for cellular differentiation and is utilized also for repressions of the transposable elements (TE's) that have become part of the human genome limits efficiency and accuracy of repair of the damages to DNA and of the damages to a subset of chromatin-nuclear lamina-envelope proteins.
(ii) Terminally differentiated cells in organs comprised of predominantly such cells and also the cells capable of proliferation, including the stem cells in adult tissues, show increases of unrepaired DNA damages and somatic mutations and show also chromatin structure features and molecular markers that indicate failing attempts of repair of the damages in genetic material with increasing age of human. The age associated increases of somatic mutations and of unrepaired damages are found predominantly in heterochromatin, in accord with the constraints that heterochromatin structure places on the efficiency and accuracy of repair of the damages to genetic material.
(iii) Damages to genetic material can come from multiple sources, some practically inescapable, but do not necessarily lead to aging as testified e.g. by the prokaryotes that can be exposed to magnitudes of greater amounts of DNA damaging agents than unicellular eukaryotes without a limitation of clonal lifespan. The oxidative energy metabolism that currently exists creates a constant source of oxidative damage to the genetic material and the oxidatively damaged DNA and protein molecules increase in amount in cells in diverse tissues and organs of aging human.
(iv) Mechanisms of meiotic rejuvenation that have been identified and pointed indicate that the germline cells progressing towards meiosis and undergoing meiosis as well as the early embryo cells that the fertilized oocyte gives rise show decondensation and remodelings of chromatin that enable efficient repair of the damages in genetic material and that these are critical for the meiotic rejuvenation. Oocytes have in addition potent means of prevention of oxidative damage to the genetic material besides for repair and the fertilized oocyte enables remodeling and repairs of the paternal genetic material that can acquire oxidative damage during the movements of sperm energized by respiring mitochondria. The primordial germ cells (PGC's) are specified early during development, allowing avoidance of the damages that the later progressively differentiating somatic cells can have, and the DNA methylation is also erased during progress of PGC's to meiosis in association with the decondensation of chromatin and repair of DNA. The DNA of currently activatable TE's may exceptionally remain methylated and opportunities that TE's may have for transcription during decondensation of chromatin in germline are countered by posttranscriptional defenses as well. Thus the pointed out critical mechanisms of meiotic rejuvenation further accord with the decisive upstream mechanisms of aging that are evidenced in description.
(v) Description guides also with regard to the age-associated events observed in human and other organisms and proposed in the field as primary causes of aging but may not be so. Linear DNA molecules of eukaryotic chromosomes require specialized means of replication of their ends (telomeres) against shortening with each replication of DNA. Normal proliferative tissue cells have in general been observed to show shortening of telomeres with increasing age of human and gradual shortenings of telomeres are observed also during in vitro aging of cells whereas the prokaryotes having circular chromosomal DNA do not have a problem of replication of DNA ends and do not show a limitation of clonal lifespan. It is pointed out that the shortenings of ends of chromosomes during aging are rather consequences of the upstream events of aging that are evidenced in description.
(vi) Unicellular eukaryotes living in anaerobic environments and undergoing meiosis by uses of enzymes and other proteins that are conserved in the rest of eukaryotes signify utility of meiosis in achievements of repairs of also of non-oxidative damages to the genetic material. TE's can cause damaging of genetic material in anaerobic as well as aerobic eukaryotes and the heterochromatinization of chromatin of TE sequences serves as a defense against TE's. Yet both the 5mC modification of cytosines, employed to affect heterochromatinization of TE sequences and of host genes, and the limitations of accessibility of DNA in heterochromatin can act as sources of unrepaired damage and mutations by the mechanisms pointed out in description. On the other hand TE's are ancient with numerous families also in the prokaryotes today and neither the TE's nor 5mC modification of DNA that can occur also 30 in prokaryotes cause a limitation to the clonal lifespan of prokaryotes, further according with the decisive role in aging by the upstream mechanisms described herein.

Examples are included below that illustrate certain aspects and embodiments of the invention presented herein. Equivalents to them can be recognized and can be ascertained by not more than routine experimentation by those skilled in the field.

### Example 1. Human Cells Engineered for Intervention with Aging and Disorders of Aging of Human

Previously unknown facts in a field of science and technology are in general determined by experimental testing of hypotheses where appropriate laboratory and further resources exist for the testing. The testing and analyses of the resulting data may indicate whether a hypothesis stands or falls or may be revised according to the data and a working hypothesis about a complex problem may get developed by a scientist through tests of different predictions of it. Whereas own funds and resources of this inventor sufficed for his clinical and laboratory investigations described and referred in Example 3, the funds became inadequate later on for experimental testing of his hypotheses concerning specific interventions with aging and diseases of aging due to circumstances beyond his control (pointed in relation to Example 3 below). Testing of hypotheses can be performed on the other hand also in the absence of a laboratory facility for the intended experiments when the scientific literature and public databases have adequate relevant data. Such data may be combined from different publications and databases and may include those generated by other scientists in investigations carried out for different purposes. The solutions described in Examples 1-2 have been formulated by analyses and testing using this latter approach.

Decondensation of chromatin in PGC's, in cells in meiosis and in the early embryo cells following fertilization of oocyte contributes to the meiotic rejuvenation as it has been described above. This decondensation causes also risks of transcriptions of the TE's existing in human genome and having intact sequences for retrotransposition (the term TE, as used here, refers to the retrotransposable elements in general unless additionally specified). Transcriptions of such TE's create thereby risks of translation and retrotranspositions despite the upregulations of posttranscriptional defenses in germline. Occurrences of e.g. Alu-induced de novo mutations in germline in significant proportions of individuals today indicate in this regard that existing countermeasures against TE's in human are not fail-safe. The TE transcripts found in large quantities in early embryo cells have on the other hand been widely reported as being required for normal embryonal development and the adaptations of TE-derived sequences for uses in host functions have long been known. Uses of various zinc finger proteins for repressions of transcriptions of TE's during evolution of diverse species are known and binding of the CTCF protein to ancient TE-derived sequences with effects on physiological functions in human today is a known example of such adaptation.

I have analyzed the specific developmental events and the particular TE's described to affect them and determined that currently activatable TE's can be eliminated from human genome while having normal human cells that are capable of performing normal functions in tissues of human. Such cells can be used advantageously in comparison to their non-engineered counterparts in treatments of age-associated disorders of human as described below.

Long Interspersed Elements 1 (abbreviated hereon as LINE1), Short Interspersed Elements 1 (abbreviated hereon as SINE1/7SL; includes the Alu) and SVA composite retrotransposable elements (SINE-VNTR-Alu composites) are currently activatable non-LTR (non-long terminal repeat) TE's in human genome and the present day human genome has also LTR-containing endogenous retrovirus sequences (human ERV's; abbreviated hereon as HERV's) having open reading frames (ORF's) encoding functional retroviral proteins. Among the non-LTR TE's in human genome, LINE1 is the only autonomous currently activatable non-LTR TE; the SINE1/7SL (Alu) and SVA depend on proteins encoded by other TE's for retrotransposition and they benefit from LINE1 activity for their retrotranspositions. The HERV loci existing in the human genome today include those that encode functional viral proteins for assemblies of viral particles although most have inactivating mutations and solitary LTR sequences that have formed through deletions of LTR-flanked internal sequences of HERV's are also found in human genome. Heterochromatinization of HERV sequences in normal somatic tissue cells serves against their activation and various tumor cells have been determined to have expressions of HERV's. My investigations with normal somatic tissue cells and with their neoplastic counterparts have shown that a particular fraction of chromatin that is enriched for constitutive heterochromatin shows decondensation and increase of DNAse I accessibility of DNA during aging in normal tissue cells and that the tumor cells originating from them continue to show this age-associated modification of chromatin even though the same tumor cells do not show a disulfide-mediated condensation of chromatin structure found to occur during aging of human and of mice in normal tissue cells as it has been pointed earlier in description. Numerous investigations since then have documented activations of non-LTR TE's and of HERV's in diverse tumor cells in association with failures of maintenance of their heterochromatin structure as well as failures during aging in repressions of these TE's in normal somatic tissue cells. Taking further into account the determinations that I have pointed indicating that the heterochromatinization employed for repressions of TE's and for cellular differentiation creates conflicts with maintenance of integrity of the genome and that expressions of currently activatable TE's and their retrotranspositions can cause additional damaging of the genetic material in somatic cells to trigger apoptosis or senescence or neoplastic transformation of them, I describe herein means by which said unwanted effects are countered.

In one embodiment, the reverse transcriptase (RT) encoding sequences of the LINE1 and HERV copies that exist in the human genome and have an intact sequence to give rise to a functional RT protein are rendered incapable of giving rise to such RT by molecular genetic engineering of human cells. A functional RT encoding sequence can be rendered incapable of doing so by making relatively few nucleotide sequence changes in a RT encoding sequence, such as by changing a codon to a premature stop codon or by changing the RT amino acids essential for RT activity to result in an inactive mutant RT. Deletions of part or entirety of a RT encoding sequence of a LINE1 or HERV copy can also be performed and also provide elimination of a functional RT. Analyses of the human genome sequence reference assemblies show that out of the tens of thousands of LINE1 copies existing in human genome today, less than 200 have intact functional Open Reading Frame 2 protein (Orf2p) and Open Reading Frame 1 protein (Orf1p) encoding sequences. The Orf2p of LINE1 has RT and endonuclease domains and a CCHC type zinc finger DNA-binding domain. RT of HERV's is encoded by the pol gene of HERV (which encodes also for the integrase protein of HERV). The HERV copies in the human genome having an intact sequence encoding for a functional RT also make a small proportion of the HERV's existing in the human genome similar to the situation with the LINE1 copies. Amino acid sequences of the RT of LINE1 and of HERV and critical sequences of them for RT activity are known and sensitive methods to detect and quantify RT activity are routinely practiced in virology and in other fields of life sciences. Elimination of the entire source of the RT activity originating from the LINE1 and HERV copies existing in the human genome today can thus be readily done and verified by methods available to persons skilled in the field. The genome editing methods that have been referred earlier can be adapted specifically for eliminations of the RT proteins originating from LINE1 and HERV copies existing in the human genome. The above described complete eliminations of the functional RT proteins originating from the autonomous retrotransposons has the additional advantage of incapacitations of the remainder of the currently activatable TE's that exist in the human genome since both the SINE1/7SL (Alu) and SVA are non-autonomous and the RT supplied by the autonomous retrotransposon copies is essential for their retrotransposition. Elimination of a significant source of damaging of the genetic material from human cells without causation of adverse effects in them as described above provides multiple advantages to such engineered human cells, including longer healthy lifespan when incorporated to human tissues in vivo as described below and significantly decreased risks of undergoing neoplastic transformation in comparison to the non-engineered (wild type) human cells.

Availabilities of the encoding nucleotide sequences and of amino acid sequences of the RT's of LINE1 and of HERV's and of telomerase reverse transcriptase along with various known assays of RT activity allow setting of screens for identifications of selective small molecule inhibitors of LINE and HERV RT's that may spare the telomerase RT. Developments of beneficial treatments of particular age-associated pathological conditions by uses of pharmaceutical formulations comprising such inhibitors are suggested by the findings about them.

In another embodiment, entire sequences of all currently activatable LINE1 and HERV copies are deleted from human genome by genome editing methods that do not cause dsDNA breaks for the editing of genome of human cells. Stepwise deletions as well as multiplexing for simultaneous deletions can be performed. In genetic engineering of the human cells for therapeutic purposes, the proteins and guide RNA molecules used for the editing are introduced into cells preferably by microinjection and/or by use of liposomes containing optimized quantities of the protein and guide RNA molecules. Lengths of the currently activatable copies of LINE1 and of HERV's existing in the human genome are on average about 6 kb and 9 kb, respectively, and they amount collectively to only a few hundreds to simplify the above indicated deletions.

Deletions of entirety of the copies of LINE1, HERV, SINE1/7SL (Alu) and of SVA from human genome provide substantial reduction of the size of human genome particularly when currently non-activatable copies are also deleted. Such engineering of human genome can be carried out by using normal human embryonal stem cell (ESC) lines for optimizations of the steps and can be repeated with other cell types described below for therapeutic uses. Besides the existing human ESC lines, methods to produce new human ESC lines e.g. by uses of redundant early embryos/blastocysts that in vitro fertilization practices usually generate have been known. Human ESC lines maintained under standardized culture conditions enable detections of effects of deletions of the sequences belonging to particular TE's. Deletions not causing an undesired effect can be carried out and cataloging of the particular deletions that are observed to have an undesired effect facilitates their safe deletions by additional genetic engineering. An undesired effect of deletion of a particular TE can occur through change of promoter or enhancer function for a gene or genes e.g. when that TE is in the same DNA/chromatin loop as the gene(s). Methods to address such effects are available; e.g. addition or deletion of a CTCF target sequence and/or changing of position of such for change of loop configuration. Experiences with the much more demanding genome reduction processes involving deletions of protein-encoding sequences as well from unicellular eukaryotes that have been referred accord with achievements of deletions of the above indicated TE sequences from human genome more simply.

Human cells having substantial reduction of the size of genome through above described deletions of the currently activatable and inactive TE sequences from human genome can be advantageously used in treatments of aging human. The substantial decrease of size of human genome and of the burden of constitutive heterochromatin can provide far more effective maintenance of genome integrity than otherwise available in vivo in human treated to have such cells incorporated into tissues and organs as described below. Example 2 below describes uses of such cells and of further modified somatic cells in interventions with disorders of aging of human.

The specific genetic engineerings of human genome that are indicated above can be performed with normal somatic tissue stem cells of a particular person or patient for uses of the cells in his or her treatments. In addition, such human cells can be engineered so as to lack transplantation antigens and expanded in industrial scale with measures and quality controls against occurrences of clones showing an undesired genotype/phenotype. The produced cells of male and female sexes can be viably stored in vials or packages (e.g. in liquid nitrogen) in numbers suitable for particular therapeutic uses in treatment of any person. Genetic engineering methods that allow incorporation of desired genes to a desired position of genome of a cell are known and can be used for introduction of person-specific transplantation antigens to such industrially manufactured human cells. It provides histocompatibility with a person to be treated while avoiding killing of the cells by the host immune system, including by the natural killer (NK) cells of host that can kill histocompatibility antigens-null cells.

### Example 2. Generation of Totipotent and Pluripotent Normal Human Cells Having Improved Maintenance of Genome Integrity for Interventions with Disorders of Aging

Cells constituting a tissue of human exist at varying states of differentiation at distinct positions relative to other cells of the same lineage and of other lineages. Stem cells of adult somatic tissues are in general the least differentiated of their lineage and identifiable by expressions of particular proteins commonly at anatomically identifiable positions (niches) where they are supported by other cell types in multiple ways and normally do not proliferate except for replacements for the cells of their lineage. Relatively infrequent proliferations and less differentiated state of normal somatic tissue stem cells provide advantages to them in lessening unrepaired/misrepaired damages in their genetic material but they too show increasing frequencies of such damages and somatic mutations during aging of human with marked enrichments in heterochromatinic regions of genome in comparison to those in euchromatin. The stem cells having less genetic damage and somatic mutations than their differentiated progenies are in general preferable for the modifications described below for interventions with aging and age associated disorders. EpCAM is an example of a molecular marker of epithelial stem cells in adults (which is expressed also by spermatogonial stem cells and by ESC's) and various other markers of normal stem cells of various cell lineages as well as methods to obtain them from a person are known. Stem cells can be genetically modified for a treatment of a person in situ in vivo and can also be modified ex vivo for subsequent incorporations to tissues of a person. Normal stem cells and the cells derived from them that have undergone in vitro the particular molecular genetic modifications described below can be incorporated to desired tissues and organs of a person to be treated by several methods. Injection of such a cell to a particular tissue site can be performed by magnified viewing of tissues by use of a scopy device or an operation microscope. Such cells can be introduced to desired tissue positions also after being combined in vitro with particular niche support cells in functional three-dimensional relationships. Open surgery as well as closed surgery methods are known for incorporation of a graft to a desired tissue position of a person.

Embryonal stem cell (ESC) lines generated from inner cell mass (ICM) cells of blastocysts of human and of many other mammalian species have been available. Methods of induction of differentiation of ESC's to desired differentiated cell types as well as culture conditions that provide undifferentiated proliferation of ESC's have also been known. ICM cells are on the other hand formed after formations of the 8-cell stage cells in which significant compaction of chromatin occurred relative to the 2-cell stage cells and chromocenters became detectable. Limitations or preclusion of use of the conventionally produced ESC's for therapeutic purposes have been known, including due to arising of cell clones having unwanted mutations during expansions of ESC's, the overtaking of ESC populations by subclones having genetic and epigenetic modifications that can produce tumors and, in case of humans, host versus graft and graft versus host reactions that can occur when foreign ESC's or their differentiated progeny are introduced to a person. Generation of iPSC's by forced expressions of pluripotency conferring transcription factors in somatic cells of a person, which can supply cells avoiding the histoincompatibility barrier, have also been determined to have significant shortcomings for uses in treatment of patients. For example the somatic mutations that show increasing frequencies in cells of human during aging and enriched in heterochromatin persist in the iPSC's generated from them. I describe herein solutions to the existing problems and shortcomings and describe methods of generation of normal human cells that can be used advantageously in interventions with aging and age-associated disorders of human.

Oocytes, including the human oocytes used in clinical practices for in vitro fertilization (IVF) with sperm of a man and for intracytoplasmic injections of sperm or of round spermatids, have capabilities of provision of meiotic rejuvenation. Besides the small molecules (GSH, cysteine and others) and enzymes available in the oocyte cytoplasm for prevention of and for repair of oxidative damages to the genetic material, oocytes have also proteases, chromatin remodelers and other proteins and RNA molecules that serve for avoidance of and repairs of the damages by further sources as well (e.g. by TE's) and provide such support also to the incoming male genetic material. Methods to stimulate formations of ovarian follicles in women by administering gonadotrophins, collections of grown follicles by transvaginal aspiration, in vitro maturation of and preparation of oocytes for IVF with sperm of a man or for intracytoplasmic injection with sperm or with another cell type while visualizing under microscope have been known and widely practiced. Uses of microscopes equipped with micromanipulators and warmed stages for aspirations from an oocyte are also known. Aspiration of the meiotic spindle together with the associated oocyte chromosomes from a metaphase II stage oocyte by uses of micropipettes and introduction into an enucleated oocyte a live germline cell or a live somatic cell by various methods (e.g. by placement into perivitelline space a cell whose plasma membrane was rendered fusogenic) have also been known and practiced with oocytes and cells of diverse species including human. In cases of introduction of a normal somatic cell into a properly enucleated oocyte, causation of rapid breakdown of the nuclear envelope-lamina of the introduced somatic cell nucleus by processes employing the enzymes, other proteins and small molecules present in the oocyte cytoplasm are determined in testimony of the critical mechanisms of meiotic rejuvenation that I have pointed earlier. Remodeling of chromatin of the somatic cell in the oocyte cytoplasm and progression of the enucleated oocyte-somatic cell nucleus combination to normal cell division to produce cells like those of the 2-cell stage embryos forming by the IVF of a non-enucleated oocyte with sperm have been described with diverse mammalian species. Further developments of them to normal blastocyst-like structures (called somatic cell nuclear transfer, SCNT, embryos) have also been determined. Transfers of SCNT embryos to uterus of foster females, developments of some of them to give rise to fertile adult males and females whose genome has originated from the transferred somatic cell nucleus have also been described with diverse mammalian species albeit at very low success rates. Experimental conditions increasing the human SCNT blastocyst formation rates and allowing generation of cell lines from their ICM with gene expression patterns of conventionally produced human ECS's have been known. Such human ESC-like cells derived from human SCNT blastocysts avoid the histoincompatibility problem posed by the conventionally produced human ESC's but continue to suffer from the aforementioned shortcomings of conventional human ESC's.

Specific modifications of the SCNT blastocyst production process and additional methods are described herein that provide normal diploid totipotent and pluripotent human cells that show features of meiotic rejuvenation and can be incorporated to tissues of the person from whom somatic cells had been obtained for introduction to enucleated oocytes. The differences from previously described methods of generation of SCNT-derived embryo cells include the following. (1) Duration of incubation of the enucleated oocyte-somatic cell nucleus combination prior to the activation step (activation e.g. by pulses of direct current) is optimized, typically by prolongation in case of a combination where the somatic cell is from an older person. Somatic tissue cells of older people are found to have increases of unrepaired damages in DNA and in protein components of genetic material as it has been pointed above. Removal of spindle with the associated chromosomes of oocyte during the oocyte enucleation step is performed with attention to aspiration of minimal amount of oocyte cytoplasm during the spindle removal to avoid causation of decreases of the oocyte molecules employed for repairs of the damages in somatic cell nucleus. Damages existing in somatic cell nucleus in cases of older people, e.g. at nuclear envelope-lamina-heterochromatin components, can take longer for the enzymes and other oocyte factors to act upon. Methods to transfer cytoplasm from another oocyte to a desired oocyte are known and can be performed where such may be needed. Optimization of said period for somatic cells of older people can be readily done by persons skilled in the field in view of the specific effects described above. (2) Culture conditions of the enucleated oocyte-somatic cell nucleus and of the cells derived therefrom are optimized specifically for minimization of causation of oxidative damage by the culture conditions to cells and by provisions of culture conditions supportive to the remodeling of chromatin and timely demethylation of DNA of the somatic cell. Besides lowering of the O2 concentration in tissue culture incubator from the usual about 20% atmospheric O2 down to a physiologic level, having optimal concentrations of reducing agents, of effectors of DNA demethylation and methylation enzymes and of histone modifying enzymes (e.g. acetylating and deacetylating enzymes) in culture media can be done by concentration optimization methods generally used for other molecules. Bisulfite sequencing of DNA can be used for monitoring of the DNA methylation states of particular genome sequences, including of the sequences of imprinted genes. (3) The normal somatic cells of a person to be introduced to enucleated human oocytes are selected from the somatic tissue stem cells of that person. The normal somatic stem cells located at tissue sites where they experience relatively less damage to the genetic material and undergo relatively less proliferation during the lifetime of person are preferred. Spermatogonial stem cells (in case of a male) can provide particular advantages for introduction to enucleated oocytes. They originate from the PGC's specified early during development and have further advantageous features for maintenance of genome integrity as it has been pointed above.

In a particular embodiment, cells of the 2-cell stage or 4-cell stage produced by the above described process employing introduction of somatic tissue stem cells to enucleated human oocytes are taken from the culture dishes when they are formed and each cell of the 2-cell stage and 4-cell stage is introduced to a new enucleated human oocyte. The above described process of productions of cells of the 2-cell and 4-cell stages is then followed using these newly made enucleated oocyte-2-cell stage cell nucleus and enucleated oocyte-4-cell stage cell nucleus combinations. Reiterations of the uses of the 2-cell stage and 4-cell stage cells for the introduction of nucleus of each to a new enucleated oocyte can provide increases of the numbers of the somatic cells having advantageous meiotic rejuvenation for incorporations into tissues of the person from whom the somatic tissue stem cells had been obtained. Full histocompatibility of the produced rejuvenated cells with the existing tissue cells of a person to be treated and the large numbers of such normal cells that can be produced by the described reiterations provide advantages in interventions with aging and age-associated disorders of human.

In another embodiment, the above described reiterations of the process are done with cells obtained from the 8-cell stage to blastocyst stages of development. ICM cells of the blastocysts visualized under microscope can be dissociated and introduced without delay into enucleated human oocytes (e.g. by placement of an ICM cell into perivitelline space of enucleated oocyte after rendering the plasma membrane of the ICM cell fusogenic).

The capability of repeatedly generating own meiotically rejuvenated pluripotent normal diploid cells (as well as of their differentiated progenies that can be obtained for desired somatic tissues and organs) as described herein provides effective means of interventions with aging and age-associated disorders of human. In addition the upstream basic causes and events of aging that have been pointed out earlier are also addressed by the embodiments described below.

In a particular embodiment, the eliminations of the currently activatable TE's from human genome that have been described in Example 1 are performed for normal somatic cells of the person to be treated and such genetically engineered cells are introduced into enucleated human oocytes. The culturing and process that have been described above is then performed with such enucleated oocyte-engineered somatic cell nucleus combinations. The reiterations using the 2-cell stage and 4-cell stage cells and using the 8-cell stage to ICM stage cells that have been described above can also be done and they can provide large numbers of advantageously rejuvenated cells having also the advantage of having been rendered devoid of functional RT's of TE's. The meiotically rejuvenated engineered human cells produced as described herein provide distinct advantages in interventions with aging and age-associated disorders of human, including due to having longer healthy lifespans in tissues of treated persons and due to having significantly lowered risks of neoplastic transformation in comparison to wild type human cells. They can be used particularly advantageously in interventions with aging of CNS of human.

In a further embodiment, the industrially produced human cells that are described in Example 1 to have eliminations of currently activatable TE's from human genome and to have been rendered devoid of histocompatibility antigens so as to be suitable for use in treatments of any person by integration of the histocompatibility antigens-encoding genes of a person to the genome of such cells are used for introduction into enucleated oocytes and the meiotic rejuvenation process described above is performed with such cells. Industrial productions of said genetically engineered human cells from which currently activatable TE's and histocompatibility antigens-encoding genes have been removed can be done also by inclusion of a step in the industrial production process comprising the above-described meiotic rejuvenation process for the produced cells. These industrially produced cells can then be used for treatment of a patient by integration to the genome of such a cell the histocompatibility antigens-encoding genes of the patient.

Methods known for in vitro generation of oocytes from PGC's and from PGC-like cells can be adapted for large scale productions of oocytes suitable for enucleation for use in the above described methods of generations of meiotically rejuvenated human cells for therapeutic uses in human.

The more demanding engineering of the human genome to include tailored modifications of the protein encoding sequences of human genes to cause lowering of the rates of damaging of genetic material and to cause improvements of repairs of such damage in comparison to those in the human cells having wild type genome can also be done as it has been pointed out earlier in cells wherein less demanding significant reductions of the size of human genome have also been caused. Such cells can also be subjected to a process of meiotic rejuvenation described above and provide further advantageous human cells for interventions with disorders arising from aging of human.

### Example 3. Development of an Effective Treatment for a Frequent Age-Associated Disease May not be Adequate for its Bringing to Patients in Need: An Example Informative about Remedy

Effective new treatments for frequent diseases of aging lacking a previously known satisfactory treatment are developments disruptive to the status quo and it is pointed herein that scientific proof of an effective treatment and desirable safety do not suffice for its reach to patients under present circumstances. A new drug treatment documented to have superior therapeutic effectiveness and safety over those in practice must still go through an expensive regulatory review process before it can be introduced to market. It does not need on the other hand to have been demonstrated by a well-funded scientist. Such a treatment would normally be expected to qualify for support of public institutions chartered to promote public health and science for presentation to regulatory review. Private industry or capital might also be expected to do so in return for a share in proceeds. Yet such has not occurred in case of the new drug treatment referred below. Because the referred new drug treatment is in areas of primary research interest and expertise of this inventor and was developed through investigations he designed and participated, information about the treatment and record are presented herein with adequate detail and references for independent verifications and for checking against potential of bias.

The World Intellectual Property Organization publication WO 2018/048367 describes the above mentioned treatment which is for patients having a tumor that is not suitable for treatment by surgical excision. A new drug treatment of tumor bearing patients is described to scientists having expertise in the fields related to it. Because a complex scientific-technological matter is presented therein for experts in the field of that invention and the information in the scientific publications referred therein cover thousands of pages, here a summary is provided about salient features of the treatment and about its development in consideration of scientists whose primary expertise may not be in the field of that invention. The new drug treatment has been assessed in tumor bearing human subjects following earlier findings about the mechanisms of tumorigenesis during aging, mechanisms of avoidance of differentiation and senescence by tumor cells and concerning the mechanisms that enable tumorigenic cells to survive in tissues away from where they originate. The treatment has been determined in clinical investigations to provide rapid disappearances of tumors without recurrence independent of the histopathological class, anatomic location and invasions of tumor in the investigated cases. Pharmaceutical formulations comprised of a selective inhibitor of Hedgehog/Smoothened (Hh/Smo) signaling are administered to patients for this treatment. A related narrower scope method of treatment evaluated with patients having various skin tumors had previously been reported (Taş S, Avci O. Induction of the differentiation and apoptosis of tumor cells with efficiency and selectivity. Eur J Dermatol 2004; 14:96-102). Several other clinical trials were also reported after 2004 by different teams with patients having tumors of various organs administered with pharmaceutical formulations comprised of various selective inhibitors of Hh/Smo signaling prior to the reporting of above mentioned treatment in WO 2018/048367. Previous clinical trial reports are referred in WO 2018/048367 and differences of the tumor treatment described in WO 2018/048367 from them are pointed.

Hh/Smo signaling affects processing and cellular localizations of transcription factors Gli 1, 2, 3. The nucleotide sequences recognized and bound by Gli proteins exist at thousands of positions in human genome. Because structure of chromatin at a Gli binding site affects its availability for binding of Gli and because expressions of Hh target genes can be affected also by other transcription factors and by combinatorial effects, potential of a huge number of different responses exists to Hh in receiving cells depending on the type and life history of receiving cells. In addition, concentration of Hh and duration of exposure to Hh also affect the responses to Hh, further increasing the number of different responses in tissues. Hh/Smo signaling is necessary for vital normal functions in every person and the conditional genetic inactivations of Hh/Smo signaling in adult experimental animals have shown that it is impossible to keep adults alive in the absence of Hh/Smo signaling. Tumor cells have been reported to show increased Hh/Smo signaling activity in comparison to normal tissue cells. WO 2018/048367 describes an experimental design and methods that allow determinations of the effects of continually varying concentrations of a selective inhibitor of Hh/Smo signaling on different cell types and tissue structures simultaneously in their natural environments in vivo in human. Determinations of the effects of varying doses of a selective inhibitor of Hh/Smo signaling on normal cell types and on tumor cells simultaneously are described. Details of the testing results and the insights provided by them, which are not possible to obtain by conventional pharmacological methods and by conventional clinical testing for dose finding for a candidate drug molecule, are described along with the uses of the findings for treatment of tumor bearing human.

Results of clinical investigations are described showing that a selective inhibitor of Hh/Smo signaling exerts different dose-dependent effects of on normal cells and on tumor cells. In case of tumor cells, an inhibition of proliferation is observed with gradual increase of dose and the tumor cells showing inhibition of proliferation are found to show further dose dependent effects: such a tumor cell can remain undifferentiated and can resume proliferation later on but with further increasing of exposure to medicament the tumor cells are induced to differentiate at unusually high frequencies for an in vivo effect. It is also shown that the tumor cells can be eliminated rapidly by induction of apoptosis of them with high efficiency within a window of exposure that is above that suffices for induction of differentiation of the same tumor cells. WO 2018/048367 describes that not only the amount of a selective inhibitor of Hh/Smo signaling to which tumor cells are exposed but the time frame and rate of exposure are also relevant for elimination of tumor cells from patients and that these variables are relevant also for the effects on normal cells and normal functions in patients. Lowering the amount of Hh/Smo signaling inhibitor administered in a day in order to decrease the unwanted effects on normal cells and increasing the number of days to increase therapeutic effectiveness are described to be counterproductive as revealed by the simultaneously determined effects of varying doses on normal tissue cells and on tumor cells. Normal stem cells and progenitor cells that are dependent on Hh/Smo signaling for normal functions and whose harming by selective inhibition of Hh/Smo signaling can be lethal are shown to be spared within a narrow but achievable dosing window by morphological and molecular markers criteria and by functional criteria. The latter include the long term preservations of functions known to depend on stem cells in the cases followed up for several years. WO 2018/048367 describes examples of these.

The treatment has been determined not to exert a genotoxic effect in patients. Nongenotoxicity, high efficiency and rapidity of induction of apoptosis of tumor cells and achievements of these by a tolerable dosing that allows sparing of patient's normal cells contribute to the advantageous therapeutic results and safety that have not been previously brought to tumor bearing patients. Whether or not a tumor caused to become invisible has been fully eliminated and the tumor does not show recurrence are also critical for patients treated for cancer. Previous clinical trials with tumor bearing men and women administered with various pharmaceutical formulations comprised of a selective inhibitor of Hh/Smo signaling had in general described causation of tumor non-detectability in a small minority of patients and had described recurrences of tumors and typical resistance of the recurring tumors to further treatment attempts. The treatment described in WO 2018/048367 has been determined to provide disappearances of tumors without recurrence by the stringent criterion of lack of recurrence over long-term follow up (includes follow ups for more than 7 years). Statistical analyses show that the determined causations of disappearances of tumors without recurrence cannot be due to a chance occurrence in the series so far (p<0.002) and the probability becomes practically nil when comparisons are made with results for non-treated and otherwise treated patients reviewed in scientific literature.

Investigations of the inventor of the treatment described in WO 2018/048367 have been carried out mostly by his own funds in Turkey except for the earlier investigations about mechanisms of aging, tumorigenesis associated with aging and about differences of neoplastic cells from normal cells which were carried out at University of California and University of Texas in USA during 1976-1982 and at Kuwait University during 1985-1990. Using of own funds for the investigations was necessitated because of unavailability of funding and laboratory facilities from the university where the author was a professor during 1990's and had been critical of the university administration (Taş S. Biyokimya Dergisi-Turkish Journal of Biochemistry 1998; 23:42-47 is a publication having related information). Because having to carry out scientific research with own funds unduly constrains a scientist and because collaborations with qualified willing scientists and institutions would be mutually beneficial in view of the findings reported in WO 02/078703 (PCT/TR01/00027) about a narrower scope treatment of tumor bearing human, he traveled to USA in 2001 to explore collaborations with former colleagues and scientists he knew but faced a serious criminal attack within a few days of arrival at USA that necessitated interruption of the planned explorations before they could start. Upon recovery from acute effects of the attack, he returned to Turkey where he had to continue his scientific work with own funds. The affidavit/sworn declaration (exhibit 2098) filed at US Patent and Trademark Office in relation to the interferences 105926 and 105949 concerning his U.S. Pat. No. 7,893,078 issued on basis of a US patent application with priority of PCT/TR01/00027 describes circumstances of his investigations leading to the treatment described therein. Following a nonprecedential decision of US CAFC (2015-1175) in appeals of the interferences, the matter was brought to US Supreme Court. The US Supreme Court has chosen not to make a decision in the matter by declining to review the case (2015-1089). The investigations and findings published for the first time in WO 2018/048367 have also been carried by own funds of the inventor due to unavailability of support from public and private institutions contacted. A letter published by WIPO at its website in relation to PCT/TR2017/000043 indicating the inventor's interest in collaboration and licensing agreements is an example of his attempts to get support and collaboration in bringing a described new drug treatment to regulatory approval and thereby to patients at large. Above summarized features of the newly developed treatment of tumor bearing patients are independently verifiable by scientists in its field and the above referred record and responses related to the development of a new drug treatment bringing previously unavailable solutions to a serious health problem affecting large proportions of public are open to public scrutiny. What the inventions described and referred above provide are critical in solution of a frequent problem associated with aging of human and what they have revealed may be illustrative about the shortcomings in the current economic and international systems for achievements of scientific-technological advancements. They can in addition be seen to provide opportunities for improvements.

### PUBLICATIONS REFERENCED

1. Meerding W J et al. Demographic and epidemiological determinants of healthcare costs in Netherlands: cost of illness study. BMJ 1998; 317:111-115.
2. Xu J et al. The economic burden of dementia in China, 1990-2030: implications for health policy. Bull World Health Organ 2017; 95:18-26.
3. Sado M et al. The estimated cost of dementia in Japan, the most aged society in the world. PLOS One 2018; 13; e0206508.
4. Smith T M et al. Dental evidence for ontogenetic differences between modern humans and Neanderthals. Proc Natl Acad Sci USA 2010; 107:20923-20928.
5. Finch C E, Austad S N. Primate aging in the mammalian scheme: the puzzle of extreme variation in brain aging. Age 2012; 34:1075-1091.
6. Taş S. Intervention with disorders of aging. WO 2019/135727 (World Intellectual Property Organization, Geneva, Switzerland) (2019).
7. Ikeno Y et al. Do Ames dwarf and calorie-restricted mice share common effects on age-related pathology? Pathobiol Aging Age Relat Dis 2013; 3:20833.
8. Mattison J A et al. Caloric restriction improves health and survival of rhesus monkeys. Nat Commun 2017; 8:14063.
9. Pifferi F et al. Promoting healthspan and lifespan with caloric restriction in primates. Commun Biol 2019; 2:107.
10. Auton A et al (The 1000 Genomes Project Consortium). A global reference for human genetic variation. Nature 2015; 526:68-74.
11. Okumura T, Sawamura A, Murohara T. Palliative and end-of-life care for heart failure patients in an aging society. Korean J Intern Med 2018; 33:1039-1049.
12. Taş S. Cellular aging, neoplastic transformation, meiotic rejuvenation, and the structure of chromatin complex. Cellular Ageing (Karger, Basel, Switzerland) pp. 178-192 (1984). 13. Hedges S B et al. A genomic timescale for the origin of eukaryotes. BMC Evol Biol 2001; 1:4.
14. Makarova K S et al. Genome of the extremely radiation-resistant bacterium Deinococcus radiodurans viewed from the perspective of comparative genomics. Microbiol Mol Biol Rev 2001; 65:44-79.
15. Taş S, Walford R L. Disulfide bonds and the structure of the chromatin complex in relation to aging and spontaneous malignancies of old age. Age 1980; 3:95.
16. Taş S, Tam C F, Walford R L. Disulfide bonds and the structure of the chromatin complex in relation to aging. Mech Ageing Dev 1980; 12:65-80.
17. Taş S, Walford R L. Influence of disulfide reducing agents on fractionation of chromatin complex by endogenous nucleases and DNAse I in aging mice. J Gerontol 1982; 37:673-679.
18. Taş S, Walford R L. Increased disulfide mediated condensation of the nuclear DNA-protein complex in normal lymphocytes during postnatal development and aging. Mech Ageing Dev 1982; 19:73-84.
19. Taş S et al. Consistently greater decondensation of the nuclear DNA-protein complexes from normal lymphocytes than from acute and chronic lymphocytic leukemia cells following treatment with disulfide reducing agents. Cytologia 1985; 50:405-415.
20. Liu L et al. An integrated chromatin accessibility and transcriptome landscape of human pre-implantation embryos. Nat Commun 2019; 10:364.
21. Hatanake Y et al. Histone chaperone CAF-1 mediates repressive histone modifications to protect preimplantation mouse embryos from endogenous retrotransposons. Proc Natl Acad Sci USA 2015; 112:14641-14646.
22. Boskovic A et al. Higher chromatin mobility supports totipotency and precedes pluripotency in vivo. Genes Dev 2014; 28:1042-1047.
23. Lander E S et al. Initial sequencing and analysis of the human genome. Nature 2001; 409:860-921.
24. Peaston A E et al. Retrotransposons regulate host genes in mouse oocytes and preimplantation embryos. Dev Cell 2004; 7:597-606.
25. Hajkova P et al. Genome-wide reprogramming in the mouse germline entails the base excision pathway. Science 2010; 329:78-82.
26. Wang L et al. Programming and inheritance of parental DNA methylomes in mammals. Cell 2014; 157:979-991.
27. Hill P W S et al. Epigenetic reprogramming enables the transition from primordial germ cell to gonocyte. Nature 2018; 555:392-396.
28. Tharp M E, Malki S, Bortvin A. Maximizing the ovarian reserve in mice by evading LINE-1 toxicity. Nat Commun 2020; 11:330.
29. Xu Z et al. H2B ubiquitination regulates meiotic recombination by promoting chromatin relaxation. Nucleic Acids Res 2016; 44:9681-9697.
30. Wossidlo M et al. Dynamic link of DNA demethylation, DNA strand breaks and repair in mouse zygotes. EMBO J 2010; 29:1877-1888.
31. Lord T et al. Fertilization stimulates 8-hydroxy-2′-deoxyguanosine repair and antioxidant activity to prevent mutagenesis in embryo. Dev Biol 2015; 406:1-13.
32. Ernst C, Odom D T, Kutter C. The emergence of piRNAs against transposon invasion to preserve mammalian genome integrity. Nat Commun 2017; 8:1411.
33. King G A et al. Meiotic cellular rejuvenation is coupled to nuclear remodeling in budding yeast. eLife 2019; 8: e47156.
34. Toyama B H et al. Visualization of long-lived proteins reveals age mechanism within nuclei of postmitotic cells. J Cell Biol 2019; 218:433-444.
35. Kim J Y et al. A metabolic strategy to enhance long-term survival by Phx1 through stationary phase-specific pyruvate decarboxylases in fission yeast. Aging 2014; 6:587-601.
36. Bohnert K A, Kenyon C. A lysosomal switch triggers proteostasis renewal in the immostal C. elegans germ lineage. Nature 2017; 551:629-633.
37. Petrova B et al. Dynamic redox balance directs the oocyte-to-embryo transition via developmentally controlled reactive cysteine changes. Proc Natl Acad Sci USA 2018; 115, E7978-7986.
38. Blokzijl F et al. Tissue-specific mutation accumulation in human adult stem cells during life. Nature 2016; 538:260-264.
39. Shimoda N et al. Decrease in cytosine methylation at CpG island shores and increase in DNA fragmentation during zebrafish aging. Age 2014; 36:103-115.
40. Zhou W et al. DNA methylation loss in late-replicating domains is linked to mitotic cell division. Nat Genet 2018; 50:591-602.
41. Malik S B et al. An expanded inventory of conserved meiotic genes provides evidence for sex in Trichomonas vaginalis. PLOS One 2008; 3: e2879.
42. Ehrenkaufer G M et al. The genome and transcriptome of the enteric parasite Entamoeba invadens, a model for encystation. Genome Biol 2013; 14: R77.
43. Nakamichi N et al. His-to-Asp phosphorelay circuitry for regulation of sexual development in Schizosaccharomyces pombe. Biosci Biotechnol Biochem 2002; 66:2663-2672.
44. Rohme D. Evidence for a relationship between longevity of mammalian species and lifespans of normal fibroblasts in vitro and erythrocytes in vivo. Proc Natl Acad Sci USA 1981; 78:5009-5013.
45. Sadaie M, Maito T, Ishikawa F. Stable inheritance of telomere chromatin structure and function in the absence of telomeric repeats. Genes Dev 2003; 17:2271-2282.
46. Surralles J et al. Accelerated telomere shortening in the human inactive X chromosome. Am J Hum Genet 1999; 65:1617-1622.
47. Machiela M J et al. Female chromosome X mosaicism is age-related and preferentially affects the inactivated X chromosome. Nat Commun 2016; 7:11843.
48. Taş S, De Larco J, Altiok E. Agarose gel electrophoretic evidence for domains of nuclear DNA linked with bonds cleavable with sulfhydryl molecules. FEBS Lett 1985; 191:136-140.
49. Taş S. Separation of the DNA molecules beyond conventional size limits by gel electrophoresis with sodium dodecyl sulfate. Anal Biochem 1990; 188:33-37.
50. Uusküla-Reimand L et al. Topoisomerase II beta interacts with cohesin and CTCF at topological domain borders. Genome Biol 2016; 17:182.
51. Hoa N N et al, Mrell is essential for the removal of lethal topoisomerase 2 covalent cleavage complexes. Mol Cell 2016; 64:580-592.
52. Campbell R R, Wood M A. How the epigenome integrates information and reshapes the synapse. Nat Rev Neurosci 2019; 20:133-147.
53. Le Gros M A et al. Soft X-ray tomography reveals gradual chromatin compaction and reorganization during neurogenesis in vivo. Cell Rep 2016; 17:2125-2136.
54. Abascal F et al. Somatic mutation landscapes at single-molecule resolution. Nature 2021; 593:405-410.
55. Klein H U et al. Epigenome-wide study uncovers large-scale changes in histone acetylation driven by tau pathology in aging and Alzheimer's human brains. Nat Neurosci 2019; 22:37-46.
56. Shanbhag N M et al. Early neuronal accumulation of DNA double strand breaks in Alzheimer's disease. Acta Neuropathol Commun 2019; 7:77.
57. Shibata H et al. In vivo reprogramming drives Kras-induced cancer development. Nat Commun 2018; 9:2081.
58. Li H et al. The Ink4/Arf locus is barrier for iPS cell reprogramming. Nature 2009; 460:1136-1139.
59. Hikabe O et al. Reconstitution in vitro of the entire cycle of the mouse female germ line. Nature 2016; 539:299-303.
60. Taş S. Medicaments and uses in treatment of cancer and other pathological conditions associated with aging. WO 2018/0483367 (World Intellectual Property Organization, Geneva, Switzerland) (2018).
61. Ozaki Y et al. Inter- and intramolecular disulfide bond formation and related structural changes in the lens proteins. A Raman spectroscopic study in vivo of lens aging. J Biol Chem 1987; 262:15445-15551.
62. Fan X et al. Evidence of highly conserved β-crystallin disulfidome that can be mimicked by in vitro oxidation in age-related human cataract and glutathione depleted mouse lens. Mol Cell Proteomics 2015; 14:3211-3223.
63. Borchman D, Stimmelmayr R, George J C. Whales, lifespan, phospholipids, and cataracts. J Lipid Res 2017; 58:2289-2298.
64. Christen W G et al. Age-related cataract in men in the selenium and vitamin E cancer prevention trial eye endpoints study: a randomized clinical trial. JAMA Ophtalmol 2015; 133:17-24.
65. King D A. The scientific impact of nations. Nature 2004; 430:311-316.
66. Xie Y, Zhang C, Lai Q. China's rise as a major contributor to science and technology. Proc Natl Acad Sci USA 2014; 111:9437-9442.
67. Smith R. Peer review: a flawed process at the heart of science and journals. J R Soc Med 2006; 99:178-182.
68. Anderson M S et al. The perverse effects of competition on scientists' work and relationships. Sci Eng Ethics 2007; 13:437-461.
69. Iyengar S, Massey D S. Scientific communication in a post-truth society. Proc Natl Acad Sci USA 2019; 116:7656-7661.
70. Massey D S. A brief history of human society: The origin and role of emotion in social life. Am Soc Rev 2002; 67:1-29.
71. Treisman D. The causes of corruption: a cross-national study. J Public Econ 2000; 76:399-457.
72. Grier R. The effect of religion on economic development: A cross national study of 63 former colonies. Kyklos 1997; 50:47-62.
73. Maoz Z, Henderson E A. The world religion dataset, 1945-2010: Logic, estimates and trends. Int Interact 2013; 39:265-291.
74. Kouvonen A et al. Organisational justice and smoking: the Finnish public sector study. J Epidemiol Community Health 2007; 61:427-433.
75. Kobayashi Y, Kondo N. Organizational justice, psychological distress and stress-related behaviors by occupational class in female Japanese employees. PLOS One 2019; 14: e0214393.
76. Lee J et al. Abrogation of HLA surface expression using CRISPR/Cas genome editing: a step toward universal T cell therapy. Sci Rep 2020; 10:17753.
77. Anzalone A A et al. Search-and-replace genome editing without double-strand breaks or donor DNA. Nature 2019; 576:149-157.
78. Li J et al. Structure-guided engineering of adenine base editor with minimized RNA off-targeting activity. Nat Commun 2021; 12:2287.
79. Smith C J et al. Enabling large-scale genome editing at repetitive elements by reducing DNA nicking. Nucleic Acids Res 2020; 48:5183-5195.
80. Li J et al. Systematic analysis reveals the prevalence and principles of bypassable gene essentiality. Nat Commun 2019; 10:1002.
81. Luo Z et al. Compacting a synthetic yeast chromosome arm. Genome Biol 2021; 22:5.

The referencing to a publication here is intended to incorporate its descriptions as prior art for the descriptions herein.

1. A genetically engineered cell,
wherein the cell is derived from a cell isolated from a human subject and genome of the cell is engineered in such a way that the cell is rendered devoid of and incapable of having reverse transcriptase activity provided by the reverse transcriptase proteins encoded by the Long Interspersed Elements 1 and Human Endogenous Retrovirus classes of transposable elements existing in human genome.

2. A genetically engineered cell according to claim 1, wherein the Long Interspersed Elements 1 and Human Endogenous Retrovirus copies existing in the human genome are rendered incapable of encoding a functional reverse transcriptase protein by a change of their nucleotide sequences that causes a premature stop codon that precludes biosynthesis of a functional protein or by a deletion of part or entirety of their reverse transcriptase encoding sequences or by a change of their nucleotide sequences that causes an amino acid sequence change that causes loss of reverse transcriptase activity.

3. A genetically engineered cell according to claim 1, wherein the cell has deletions of the genome sequences that encode for the human transplantation antigens or has in addition integration to its genome of the sequences encoding personal histocompatibility antigens of a person to whom the cell is to be transplanted.

4. A genetically engineered cell according to claim 1, wherein the cell has additionally deletions of one or more copies of a transposable element that belongs to a Short Interspersed Elements and/or a Long Interspersed Elements and/or a SVA and/or a Human Endogenous Retrovirus class.

5-7. (canceled)

8. A process of production of diploid normal human cells,
wherein
(i) a normal somatic cell of a human is genetically engineered in such a way that the cell is rendered devoid of and incapable of having reverse transcriptase activity provided by the reverse transcriptase proteins encoded by the Long Interspersed Element 1 and Human Endogenous Retrovirus classes of transposable elements existing in human genome,
(ii) nucleus of the cell is introduced into an oocyte from which the oocyte spindle and associated oocyte chromosomes have been removed,
(iii) the resulting enucleated oocyte-engineered somatic cell nucleus construct is cultured to give rise to 2-cell stage cells and to cells of subsequent stages that may include the blastocyst-inner cell mass stage and
the diploid cells produced by the process are viably stored for a therapeutic use.

9. A process according to claim 8, wherein a cell produced at 2-cell stage or a subsequent stage is taken and its nucleus is introduced into a new enucleated oocyte and reiterations of the process are performed that provide increase of number of the diploid normal human cells that have been subjected to meiotic rejuvenation for said use.

10. A process according to claim 8, wherein the process includes at step (i) deletion from genome of said normal somatic cell of one or more copies of a transposable element that belongs to a Short Interspersed Elements and/or Long Interspersed Elements and/or SVA and/or Human Endogenous Retrovirus class.

11. A process according to claim 8 or claim 9 or claim 10, wherein the process includes at step (i) of claim 8 rendering of the cell devoid of histocompatibility antigens and integration, after completion of the production of the cells at step (iii) of claim 8, the histocompatibility antigens encoding genes of a human subject who is to be treated by use of the produced cells.

12-13. (canceled)

14. A cell derived from a genetically engineered cell of claim 1 or claim 2 or claim 3 or claim 4, wherein the cell is derived in a process comprising
(i) introduction of the nucleus of a cell of any one of the claims 1 to 4 into cytoplasm of an enucleated oocyte in vitro, and
(ii) production of two or more cells in vitro by use of said construct of enucleated oocyte-genetically engineered cell nucleus, and
(iii) introduction of the nucleus of a cell produced at step (ii) into a new enucleated oocyte cytoplasm and production of two or more cells by use of said new enucleated oocyte-genetically engineered cell nucleus construct.

15. (canceled)

16. A therapeutic product for use in treatment of a human subject,
wherein the product comprises a cell produced as specified in claim 8 or in claim 9 or in claim 10 and/or a cell of a differentiated progeny of a cell produced as specified in claim 8 or in claim 9 or in claim 10, and
wherein the treatment comprises incorporation of one or more cells of said product into a tissue or organ site of human subject.

17. A therapeutic product for use in treatment of a human subject,
wherein the product comprises a cell produced as specified in claim 11 and/or a cell of a differentiated progeny of a cell produced as specified in claim 11, and
wherein the treatment comprises incorporation of one or more cells of said product into a tissue or organ site of the human subject.

18. A therapeutic product for use in treatment of a human subject,
wherein the product comprises a cell genetically engineered according to claim 1 or claim 3 or claim 4, and/or a cell which is a differentiated progeny thereof,
wherein the cell is viably stored and the treatment comprises incorporation of one or more cells of the product into a tissue or organ site of human subject.

19. A therapeutic product according to claim 18, wherein the genetically engineered cell is subjected to a process comprising
(i) introduction of the nucleus of the cell into cytoplasm of an enucleated oocyte in vitro, and
(ii) production of two or more cells in vitro by use of said construct of enucleated oocyte-genetically engineered cell nucleus, and
(iii) introduction of the nucleus of a cell produced at step (ii) into a new enucleated oocyte cytoplasm and production of two or more cells by use of said new enucleated oocyte-genetically engineered cell nucleus construct.

20. A method for treatment of a human subject,
comprising incorporation of one or more cells that are produced by a process of claim 11 into a tissue or organ of human subject.
